(19) United States
(12) Patent Application Publication
(10) Pub. No.: US 2009/0137678 A1
(43) Pub. Date: May 28, 2009

(54) HIGH YIELD SYNTHESIS METHODS FOR PRODUCING ORGANIC SALTS OF STRONTIUM

(76) Inventors: Stephan Christgau, Gentofte (DK); Jens E.T. Andersen, Vedbaek (DK)

Correspondence Address: JONES DAY, 222 EAST 41ST ST, NEW YORK, NY, 10017, US

(21) Appl. No.: 11/994,695

(22) PCT Filed: Jul. 5, 2006
(86) PCT No.: PCT/DK2006/000396

§ 371 Date: Oct. 20, 2008

(30) Foreign Application Priority Data
Jul. 6, 2005 (DK) PA 2005 00990
Dec. 16, 2005 (DK) PA 2005 01779

(51) Int. Cl. A61K 31/192; C07C 65/10; C07C 57/00; A61K 31/191; C07C 63/04; A61P 19/08; A61P 19/10

(52) U.S. Cl. 514/568; 562/477; 562/594; 514/574; 562/488

(57) ABSTRACT

New organic salts of strontium and methods of synthesizing such salts with high purity, high yields and with short processing times, at neutral conditions and at low reaction temperature, such as a temperature at or below 50° C.

### FIELD OF THE INVENTION

The present invention relates to new organic salts of strontium and to methods of synthesizing such salts with high purity, high yields and with shorter processing times than has previously been possible.

### BACKGROUND OF THE INVENTION

Alkaline earth metals and alkali metals are almost invariably found in an oxidized state as a component of metal-organic salts due to the highly reactive nature of such elements. Salts of such metal-ions are widely distributed throughout nature. Strontium is one of the less common of these elements, but is an important component of some salts due to the beneficial actions of strontium in biologic systems. Thus, efficient manufacture of very pure organic salts of strontium is of great commercial interest.

Manufacture of strontium salts having high purity and composed of organic counter-ions not found in nature is generally made by various aqueous processes and it can often be difficult to control the homogeneity and purity of the reaction products necessitating re-crystallizations and other purification steps to separate the desired strontium salt from other potential contaminants from the main group II of the periodic system, or introduced by degradation of the anion obtained during the manufacture and/or purification steps. In turn this is likely to result in low yields of the desired salt.

Other strontium salts of commercial relevance may be temperature- and/or pH labile, rendering an efficient manufacture of the salts difficult and time-consuming.

### SUMMARY OF THE INVENTION

The present invention discloses new organic salts of strontium and efficient methods for synthesis and isolation of such salts under mild conditions. In manufacturing methods according to the invention organic salts of strontium can be prepared with high yield and purity at low reaction temperature, such as temperature at or below 50° C., thereby enabling the manufacture of strontium salts with temperature sensitive organic anions, such as, e.g., biologically active organic molecules of relevance for pharmaceutical uses of the manufactured strontium salts.

Furthermore, the manufacturing methods disclosed here enable the synthesis to be performed at neutral conditions compatible with the manufacture of base or acid labile strontium salts. Examples are provided demonstrating the ability of the disclosed methods for synthesis of temperature sensitive strontium salts and giving guidelines for establishing the optimum reaction conditions for a given strontium salt synthesis. The synthesis allows production of some entirely new salts, where time, temperature and pH-value are key parameters of compound purity. The synthesis methods are applicable for the manufacture of most organic salts of strontium, but in particular carboxylic acid salts of strontium can be made with higher yield and purity according to the present invention than obtainable by other methods. As mentioned above, the present methods are of particular relevance for synthesis of strontium salts of temperature and/or pH labile anions, as the methods disclosed herein allows a control of reaction pH to neutral or weakly acidic conditions while maintaining a low temperature and a short processing time.

Specific examples of new strontium salts provided by the present invention are strontium malonate containing 1½ crystal water molecule (sesqui-hydrate), strontium di-L-ascorbate di-hydrate, strontium fumarate, strontium salicylate mono-hydrate, strontium succinate and strontium di-ibuprofenate di-hydrate and strontium maleate. These salts are described for the first time herein and the convenient manufacture of these previously undisclosed strontium salts of organic acids in high purity demonstrates the potentials of the disclosed manufacturing method for efficient synthesis of temperature- and or pH-labile salts of pharmaceutical relevance.

### DETAILED DESCRIPTION OF THE INVENTION

### Strontium

Strontium is found naturally exclusively as a non-radioactive stable element. Twenty-six isotopes of strontium have been described, but only stable non-radioactive strontium is found on earth.

In nature, strontium is practically always found in the oxidized state as a di-cation and consequently is found as a salt, complexed with inorganic anions such as carbonate, sulphate and phosphate. A relatively limited number of strontium salts have been subjected to detailed chemical characterization, with full resolution of structure and chemical properties.

Organic strontium salts have been described, but literature reports of this type of compounds are limited to rather few substances. All previously disclosed metal organic strontium containing compounds are strontium salts of anions containing carboxylic acids. The physiochemical properties of organic strontium salts have been reported to be similar to the corresponding magnesium, calcium and barium salts (Schmidbaur H et al. Chem Ber. (1989) 122: 1433-1438). Strontium salts of carboxylic acids are crystalline non-volatile solids with strong electrostatic forces holding the ions in the crystal lattice. Most crystalline forms of organic strontium salts contain various amounts of crystal water, which serves to coordinate with the strontium ions in the crystal lattice. The temperature required for melting these salt are most often so high, that before it can be reached the carbon-carbon bonds of the organic anion breaks and the molecule decomposes, generally at a temperature of 300-400° C.

### Properties of Carboxylic Acid Salts of Strontium

Carboxylic acids salts of divalent earth metals such as strontium, and especially di-carboxylic acids have some unique properties, as they can have a partial chelating effect in solution. In these cases the salt exists in solution as a complex in which the divalent metal ion is bound in a complex to the carboxylic groups of the anion. Such complexation may be important in biological systems, where the alkaline earth metals, especially calcium and magnesium, play vital physiological roles. A majority of divalent metal ions may exist in complex bound form in the aqueous environment in biological systems, rather than in a free and un-bound ionic form. Complex formation constants with the alkaline earth metals in aqueous solution are higher for amino acids than for hydroxy-carboxylic acids and the related non-carboxylic acids, which suggest that the amino group may play a role in the complex formation. Generally, the differences in association constants and hydration enthalpy for the various ligands become smaller as the radius of the metal increases. Thus, the stability of strontium complexes with di-carboxylic acid is lower than the stability of the comparable complexes with calcium and magnesium. This means that in aqueous solutions the chelating di-carboxylic acids will have a propensity to preferentially bind calcium and magnesium rather than the larger ions of strontium and barium.

Few organic strontium salts have found commercial applications, and thus no such compounds are available in large-scale chemical manufacture (>1000 kg batch size). However, recently, the strontium salt of the tetra-carboxylic acid, ranelate, has been developed for pharmaceutical use in treatment of metabolic bone diseases such as osteoporosis.

### Synthesis of Carboxylic Acid Salts of Strontium

Organic-strontium salts of carboxylic acid anions can be synthesized by a number of different pathways. A conventional method for preparation of such organic strontium salts is to utilize the reaction between an organic acid and strontium hydroxide in an aqueous solution. As an example, the reaction scheme below shows this neutralization reaction of malonic acid and strontium hydroxide salt:

Sr²⁺(aq)+2OH⁻(aq)+C₃H₄O₄(aq)→Sr(C₃H₂O₄)(aq)+2H₂O(l) Equation 1

After the reaction, which occurs rapidly upon dissolution of the solids, the suspension of dissolved strontium malonate can then be induced to precipitate by evaporation of water and subsequent concentration of the salt above the aqueous solubility of the given salt. At concentrations at or above 1.6 g/l, crystals of strontium malonate will slowly form and precipitate from the solution.

By this method, re-crystallizations are most likely to be required in order to obtain the desired strontium salt in sufficiently pure form. In turn the yield will decrease as a consequence of loss of material during re-crystallization owing to the lack of complete precipitation of strontium from solution and from formation of strontium carbonate that precipitate and due to the very low solubility of metal carbonates makes the precipitated strontium unavailable for further reaction.

The present inventors have found that a more suitable method for producing strontium salts is to utilize the neutralization reaction of the appropriate acid by strontium carbonate (Method A according to the invention—see Equation 2 below). The reaction of Equation 2 below exemplifies the most straightforward method of synthesis of the desired product and the yield may be increased by slightly heating the solution to temperatures between 20° C. and 50° C. However, this synthesis method can also be performed at lower temperatures, even temperatures down to 5° C., and thus it is particularly well suited for the productions of strontium salts of temperature-sensitive anions. The reaction given in Equation 2 can be controlled to avoid alkaline conditions, as SrCO₃ is a weak base, and carbonate is continuously removed during the reaction.

Equation 2 is exemplified by the production of strontium fumarate (2a) and strontium L-ascorbate (2b), but this is merely meant as an illustration of the reaction. Thus, the synthesis method is well-suited to alkaline labile anions. Both the ability of the reaction to occur at low temperature as well as neutral conditions may be of key importance for production of strontium salts of many important salts, such as strontium L-ascorbate and strontium acetyl-salicylate, as these anions may decompose by elevated temperature or by alkaline hydrolysis. The evolution of gas (Equation 2) indicates the progress of reaction, and the completion of reaction is identified by a stop in effervescence. The continuous removal of gaseous carbon dioxide drives the reaction to completion and ensures a high yield of the desired strontium salt.

$$\begin{matrix}Equation 2 & \\ {SrCO}_{3} ( s ) + {C}_{3} {H}_{2} {{O}_{4} ( aq )}^{2 -} + 2 {H}^{+} {}^{water} Sr ( {C}_{3} {H}_{2} {O}_{4} ) ( aq ) + {H}_{2} O ( l ) + {CO}_{2} ( g ) & ( 2 a ) \\ {SrCO}_{3} ( s ) + 2 {C}_{6} {H}_{7} {{O}_{6} ( aq )}^{-} + 2 {H}^{+} {}^{water} {Sr ( {C}_{6} {H}_{7} {O}_{6} )}_{2} ( aq ) + {H}_{2} O ( l ) + {CO}_{2} ( g ) & ( 2 b )\end{matrix}$$

By employing the reactions scheme listed in Equation 2, strontium salts of temperature sensitive anions can be produced with a higher yield and purity and without damaging the anions.

The present inventors have found that the ratio between the positive charges of strontium and the negative charges of the anion(s) should be as close as possible to 1:1, where the negative charges refers to the actual number of de-protonated acid groups on the anion(s) at the conditions employed for the crystallization reaction according to the invention. I.e. if the organic acid is mono-protonated (such as, e.g. ibuprofenate or ascorbate), two molecules of organic acid will be needed per strontium molecule in order to give a 1:1 charge ratio. However, if the organic is di-protonated (such as, e.g., malonate and salicylate) only one molecule of organic acid will be needed per molecule of strontium in order to give a 1:1 ratio between the charges of strontium and the organic acid.

More specific, Method A according to the invention comprises reacting strontium carbonate with the proper organic acid (anion) in an aqueous medium at a temperature of about 50° C. or less, such as, e.g. about 40° C. or less, about 30° C. or less, about 25° C. or less, about 20° C. or less, or about 15° C. or less for a time period of at the most about 300 min such as, e.g., at the most about 240 min, at the most about 180 min or at the most about 120 min.

The reaction may be performed between an organic acid dissolved in aqueous solution as a free acid and strontium carbonate, which is added slowly in solid form under vigorous stirring and/or mixing.

In order to avoid elevations in pH, and to accommodate the manufacture of strontium salts of pH labile anions, the reaction may be performed with continuous monitoring of the reaction vessel in order to maintain pH in the reaction vessel below about pH 9.5, such as, e.g. below about pH 9, below about pH 8.5, below about pH 8, below about pH 7.5, below about pH 7, below about pH 6.5 or below about pH 6.

Furthermore, in a method according to the invention, the maintenance of the above mentioned pH-values may improve the equilibrium conditions of Equation 2 in favor of formation of the desired organic salts of strontium. The process of the reaction described in Equation 2 is among other parameters driven by the continuous removal of carbonate as gaseous carbon dioxide. The presence of hydroxide ions will decrease the formation of carbon dioxide and is therefore less favorable.

Examples of specific strontium salts prepared by Method A are: strontium malonate with 1½ molecules of water (sesquihydrate), strontium di-ibuprofenate di-hydrate, strontium di-L-ascorbate di-hydrate, strontium fumarate, strontium salicylate mono-hydrate and strontium succinate. The terms strontium ibuprofenate di-hydrate and strontium di-ibuprofenate has been used interchangeable herein even though the term strontium di-ibuprofenate di-hydrate is the most correct.

Other strontium salts of temperature/pH-sensitive anions according to the invention may be produced by a method denoted herein as Method B. In this approach the sodium or potassium salt of the appropriate carboxylic acid anion is reacted with strontium chloride. As all organic strontium salts will be less soluble than the highly soluble chloride salt, the organic strontium salt will precipitate under these conditions leaving NaCl and excess SrCl₂ in the solution. Equation 3 below exemplifies this reaction scheme using as an example the reaction between SrCl₂ and sodium-malonate, where reaction products are added in equimolar amounts.

$$\begin{matrix}Equation 3 & \\ {SrCl}_{2} \cdot 6 {H}_{2} O ( s ) {}^{water} {Sr}^{2 +} ( aq ) + 2 {Cl}^{-} ( aq ) + 6 {H}_{2} O ( l ) \\ {C}_{3} {H}_{2} {O}_{4} {Na}_{2} ( s ) {}^{water} {C}_{3} {H}_{2} {O}_{4}^{2 -} ( aq ) + 2 {Na}^{+} ( aq ) \\ {Sr}^{2 +} ( aq ) + {C}_{3} {H}_{2} {O}_{4}^{2 -} ( aq ) -> Sr ( {C}_{3} {H}_{2} {O}_{4} ) ( aq ) & \end{matrix}$$

This method comprises reacting strontium chloride with the proper organic acid in an aqueous medium at a temperature of at the most 50° C. or less, such as, e.g. about 40° C. or less, about 30° C. or less, about 25° C. or less, about 20° C. or less, or about 15° C. or less. In the present application Method B is used for the preparation of the new salts strontium di-ibuprofenate and strontium maleate

As described above the invention provides methods for the preparation of strontium salts of temperature and/or pH sensitive anions that enables a higher yield of the desired strontium salt (compared to methods known from prior art) and at the same time keeps the formation of carbonate at a very low limit. Accordingly, the yield of the strontium salt produced by Method A or Method B may be about 70% or more, such as, e.g., about 75% or more, about 80% or more, about 85% or more, about 90% or more or about 95% or more. Furthermore, the amount of precipitated carbonate may be less than about 1%, such as, e.g., less than about 0.5% or less than about 0.2% of the amount of divalent metal salt.

In specific embodiments of the invention the anion is unstable at elevated temperatures, such as temperatures above 50° C. and/or conditions of alkaline pH, such as pH above 9.0. In this context, an anion is understood to be a molecule that can exist in a negatively charged state in an aqueous solution, and unstable is understood to mean that a quantifiable amount of said anion such as, e.g., more than 0.1%, more than 0.2% or more than 0.5% can rearrange and/or decompose and/or be subject to other modifications such as decarboxylation, dehydration, oxidations, reduction, hydrolysis, racemization and/or isomerization. Examples of anions that may be unstable under such conditions are small dicarboxylic acids (i.e. malonate, fumarate, succinate, glutarate, oxalate), β-keto carboxylic acids (i.e. acetoacetate, α-ketobutyrate, α-ketocaproirorate), α-hydroxy carboxylic acids (i.e. certain α-amino acids (leucine, glutamate) and certain aromatic carboxylic acids, where the carboxyl groups(s) are attached directly to the aromatic ring, certain complex heterocyclic carboxylic acids such as ibuprofenate and ranelate. The method described herein, utilizing a low temperature and strontium carbonate provides a very useful method for producing the desired strontium salt of decarboxylation sensitive organic anions.

As specific examples of anion instability, the present inventors have seen that strontium salts of e.g. ascorbic acid and acetylsalicylic acid decompose upon heating and forms strontium oxalate and strontium salicylate, respectively. These reactions occur at temperatures above 40-50° C. In synthesis of strontium L-ascorbate, the decomposition of the anion is readily apparent as a formation of a yellow color of the reaction mixture, with indicates the formation of degradation products of L-ascorbic acid. The new methods according to the present invention provide an efficient manufacturing method for such temperature sensitive strontium salts.

As mentioned above, the Methods A and B according to the present invention are especially well-suited for the synthesis of strontium salts of unstable or temperature-sensitive organic acids. However, in principle, the acid (anion) may be any organic acid. In specific embodiments, the organic acid is a mono-, di-, tri- or tetra-carboxylic acid. Examples of suitable organic acids for use in a method according to the invention are e.g., fumaric acid, maleic acid, malonic acid, lactic acid, citric acid, tartaric acid, oxalic acid, ascorbic acid, salicylic acid, acetyl-salicylic acid, phthalic acid, gluconic acid, L- and D-glutamic acid, pyruvic acid, L- and D-aspartic acid, ranelic acid, 2,3,5,6-tetrabromobenzoic acid, 2,3,5,6-tetrachlorobenzoic acid, 2,3,6-tribromobenzoic acid, 2,3,6-trichlorobenzoic acid, 2,4-dichlorobenzoic acid, 2,4-dihydroxybenzoic acid, 2,6-dinitrobenzoic acid, 3,4-dimethoxybenzoic acid, abietic acid, acetoacetic acid, acetonedicarboxylic acid, aconitic acid, adipic acid, alpha-ketoglutaric acid, anthranilic acid, benzilic acid, arachidic acid, azelaic acid, behenic acid, benzenesulfonic acid, beta-hydroxybutyric acid, cinnamic acid, citraconic acid, crotonic acid, cyclopentane-1,2-dicarboxylic acid, cyclopentanecarboxylic acid, cystathionine, decanoic acid, erucic acid, ethylenediaminetetraacetic acid, fulvic acid, fumaric acid, gallic acid, glucoronic acid, glutaconic acid, glutaric acid, gulonic acid, heptanoic acid, hexanoic acid, humic acid, hydroxystearic acid, isophthalic acid, itaconic acid, lanthionine, lauric acid (dodecanoic acid), levulinic acid, linoleic acid (cis,cis-9,12-octadecadienoic acid), malic acid, m-chlorobenzoic acid, melissic acid, mesaconic acid, monochloroacetic acid, myristic acid, (tetradecanoic acid), nonanoic acid, norvaline, octanoic acid, oleic acid (cis-9-octadecenoic acid), ornithine, oxaloacetic acid, palmitic acid (hexadecanoic acid), p-aminobenzoic acid, p-chlorobenzoic acid, petroselic acid, phenylacetic acid, p-hydroxybenzoic acid, pimelic acid, propiolic acid, propionic acid, p-tert-butylbenzoic acid, pyruvic acid, sarcosine, sebacic acid, serine, sorbic acid, stearic acid (octadecanoic acid), suberic acid, succinic acid, terephthalic acid, tetrolic acid, threonine, L-threonate, thyronine, tricarballylic acid, trichloroacetic acid, trimellitic acid, trimesic acid, tyrosine, ulmic acid and ibuprofenic acid.

In specific embodiments, the organic acid is an amino carboxylic acid such as, e.g., a natural or synthetic amino acid.

A particular relevant group of strontium salts are composed of strontium and anions with distinct pharmacological actions such as pharmaceutically active component is selected from the group consisting of Non Steroidal anti inflammatory agents (NSAIDs), Cyclo-oxygenase-2 (COX-2) inhibitors, COX-3 inhibitors, inducible nitric oxide synthetase (iNOS) inhibitors, PAR2 receptor antagonists, neuroleptic agents, opioids, Cyclooxygenase (COX)-inhibiting nitric oxide donators (CINOD), Disease modifying anti-rheumatic drugs (DMARD), bisphosphonates, N-acetylcholine receptor agonists, glycine antagonists, vanilloid receptor antagonists, statins, beta-blockers, neurokinin antagonists, N-Methyl-D-Aspartate (NM DA) receptor antagonists, calcitonin gene-related peptide antagonists and 6-(5-carboxy methyl-hexyloxy)-2,2-dimethyl-hexanoic acid and analogues thereof including active metabolites thereof

In specific embodiments the strontium salts according to the invention may be prepared with an anion classified as being a NSAID such as enolic acis such as piroxicam, tenoxicam and meloxicam, heteroaryl acetic acids such as diclofenac, tolmetin, ketorolac, misoprostol and zomepirac; Indole and indene acetic acids such as indomethacin, mefenamic acid, sulindac and etodolac; Para-amino phenol derivates such as phenacetin and acetaminophen; propionic acids including naproxen, flurbiprofen, fenoprofen, oxaprozin, carprofen, ketoprofen and ibuprofen; Sulphonanilides such as Nimesulide; fenamates including mefenamic acid, meclofenamate and flufenamic acid; alkanones such as nabumetome; pyrazolones including phenylbutazone, oxyphenbutazone, antipyrine, aminopyrine and kebuzone, salicylates including acetyl salicylate (aspirin), salicylate, salsalate, difunisal, olsalazine, fendosal, sulfasalazine (1,1-dimethylheptyl)-6a,7,10,10a-tetrahydro-1-hydroxy-6,6dimethyl-6H-dibenzo[b,d]pyran carboxylic acid (CT-3); thiosalicylate and paracetamol; or a pharmaceutically acceptable salt thereof.

In another embodiment of the invention the anion may be a bisphosphonate such as ibandronate, zoledronate, alendronate, risedronate, ethidronate, chlodronate, tiludronate, minodronate, incadronate, olpadronate and pamidronate.

In yet another embodiment of the invention the anion is a DMARD selected from the group consisting of doxycycline, chondroitin sulfate, methotrexate, leflounomide (ARAVA®, Aventis), dimethylnitrosamine, azatriopine, hydroxychloroqine, cyclosporine, minocycline, salazopyrine, penicillamine, aurothiomalate (gold salt), cyclophosphamide, azathioprine and pharmacologically active metabolites thereof. In yet another embodiment of the invention the anion is an inhibitor of inducible NOS (iNOS) selected from the group consisting of amino-guanidine, N^{G}-Nitro-L-arginine, N^{G}-Monomethyl-L-arginine, N⁶-(1-Iminoethyl)-L-lysine, N^{G}-Nitro-L-arginine, S-Methyl-L-thiocitrulline, N^{G}-Monomethyl-L-arginine acetate, isothiourea derivatives, such as S-methylisothiourea, S-Ethylisothiourea, S-Isopropylisothiourea, and S-(2-Aminoethyl)-isothiourea, N^{G}-Monomethyl-L-arginine acetate, 2-Iminopiperidine; 2,4-Diamino-6-hydroxy-pyrimidine; 5-chloro-1,3-dihydro-2H-benzimidazol-2-one (FR038251), 1,3(2H,4H)-isoquinoline-dione (FR038470) and 5-chloro-2,4(1H,3H)-quinazolonedione (FR191863).

Many of these compounds are unstable at elevated temperature and/or pH, and thus the synthesis methods described in the present invention provide a convenient method for their large-scale manufacture in high yield and purity.

A more detailed list of specific examples of pharmaceutically active compounds with an acid or amine group, which are suitable for use in methods according to the present invention, are: salicylates such as acetyl salicylic acid, piroxicam, tenoxicam, ascorbic acid, nystatin, mesalazin, sulfasalazin, olsalazin, glutaminic acid, repaglinid, Methotrexate, Leflounomide, Dimethylnitrosamine, azatriopine, hydroxychloroqine, cyclosporine, minocycline, salazopyrine, penicillamine, diclofenac, propionic acids such as naproxen, flurbiprofen, fenoprofen, ketoprofen and ibuprofen, pyrazolones including phenylbutazone, fenamates such as mefenamic acid, indomethacin, sulindac, meloxicam, apazone, pyrazolones such as phenylbutazone, Bisphosphonates such as zoledronic acid, minodronic acid, incadronic acid, ibandronate, alendronate, risedronate, olpadronate, chlodronate, tiludronate and pamidronate, COX-2 preferential cyclo-oxygenase inhibitors such as celecoxib, valdecoxib, etoricoxib, lumiracoxib, parecoxib, rofecoxib and deracoxib, pantotenic acid, epoprostenol, iloprost, tirofiban, tranexamic acid, folic acid, furosemide, bumetanide, kanrenoic acid, capopril, rasagiline, enalapril, lisinopril, ramipril, fosinopril, trandolapril, valsartan, telmisartan, pravastatin, fluvostatin, atorvastatin, cerivastatin, sulfadiazine, tretionin, adapalen, azelaic acid, dinoproston, levotyroxin, lityronin, doxycyclin, lymecyclin, oxytetracyclin, tetracycline, ampicilin, amoxicillin, clavulanic acid, taxobactam, nalidiksinic acid fusidinic acid and licofelone [2,2-dimethyl-6-(4-chlorophenyl)-7-phenyl-2,3,dihydro-1 H-pyrrolizine-5-yl]-acetic acid; beta Blockers such as propranolol (Inderal), atenolol (Tenormin), and pindolol (Visken), acebutolol (Sectral), bextaxolol (kerlone), bisoprolol (zebeta), carteolol (cartrol), carvedilol (coreg), esmolol (brevibloc), labetolol (normodyne), metoprolol (lopressor), nadolol (corgard), penbutolol (levatol), pindolol (visken) and propranolol (inderal), and statins such as simvastatin, mevastatin, lovastatin, atorvastatin, cerivastatin, rosuvastatin, pravastatin and fluvastatin, as well as any pharmaceutically active derivative of the compounds and.

The reaction schemes shown above (Equations 2 and 3) are depicting the final reaction for manufacture of organic strontium salts involving the commonly performed simple reaction of an inorganic strontium salt with the desired organic anion in either free acid form or available as a salt. Thus, in order to carry out these reactions it is required that the organic acid is commercially available. In the case of more complex and/or unusual anions, they will have to be synthesized prior to the preparation of the strontium salt and formation of the strontium salt by reaction schemes as outlined above may then be incorporated in the last synthesis step. In either case the methods and procedures disclosed in the present patent application may be of great use in improving the yields and purities of the desired reaction products.

All alkaline earth metal salts of carboxylic acids are soluble to some extent in aqueous solutions, but the solubility of the specific salts vary considerably depending on the size and hydrophobicity as well as electrostatic properties of the organic anion. One of the simplest organic carboxylic acids, acetate, makes well-defined crystalline salts of strontium, which are highly soluble in water (solubility 369 g/l at room temperature). Larger organic anions usually have considerable lower solubility, depending on the hydration enthalpy and lattice enthalpy of the salt. However, as various strontium salts would not necessarily form the same type of crystal structure and their crystal lattice energies are unknown, it is not possible to make theoretical calculations of the solubility of such salts, but they will have to be determined experimentally. Furthermore, a given salt may exist in different crystal structures, where important properties, such as the amount of bound crystal water varies, and thus different crystal forms will have different lattice and hydration enthalpies and thus solubility. In general crystal forms with water molecules incorporated in the crystal structure will have higher aqueous solubility that crystal forms of the same metal organic compounds with lower or no crystal-water molecules.

As an exemplification of this the present inventors describe herein as mentioned above for the first time a new crystal form of strontium malonate having 1½ water molecule bound pr. crystal unit cell (sesquihydrate, see FIG. 3). This crystalline form of strontium malonate has a higher aqueous solubility (above 2 g/l) than the previously described anhydrous strontium malonate (Briggman B & Oskarsson A 1977, Acta Cryst. B33; 1900-1906). A higher solubility may be an advantage for certain pharmaceutical formulations as it may result in a faster dissolution and dissociation of the salt when ingested orally. This new strontium malonate salt is manufactured by Method A according to the present invention, which is produced by reacting a suspension of malonic acid with strontium carbonate at a temperature maintained at or below 40° . High yield of pure strontium malonate having 1½ water molecule bound pr. crystal unit can be obtained after a reaction time of only 120 min and a single filtration step.

In general, the use of the low temperature synthesis method as describe herein may be particularly well suited to the manufacture of more hydrated forms of strontium salts having an advantage in i.e. pharmaceutical uses, due to improved dissolution and solubility.

Accordingly, in a specific embodiment of the invention, the strontium salts strontium malonate sesquihydrate, strontium di-L-ascorbate di-hydrate, strontium fumarate, strontium salicylate mono-hydrate, strontium succinate and strontium di-ibuprofenate di-hydrate and strontium maleate may be used in medicine.

However, the methods are applicable for a wide range of different strontium salts and the strontium salts generated may have various applications. Of special relevance are applications where the desired strontium salt is used in products for human use such as food-products, ingredients for pharmaceutical use, personal care products such as creams, lotions and toothpaste and vitamins and other nutritional supplements. In such cases, a high purity and homogeneous well-defined forms of the product is very important, and the manufacturing procedure described here provides a significant advantage compared to all other available methods.

The strontium salts have particular importance from a therapeutic point of view, as strontium has a proven beneficial effect on the skeletal system as well as other beneficial physiologic effects. It has been demonstrated that strontium can play a role in the skeletal system of vertebrate animals as well as in normal physiology, and that animals given strontium generally have increased bone mineralization. Also clinical investigations have been conducted with several strontium salts showing that administration of high amounts (i.e. >300 mg/day) results in elevations in bone mineral density (BMD) and thus skeletal strength. High strontium intake was in several animal studies associated with some alterations in mineralization. It has been shown that in animals subjected to longer duration strontium treatment, the hydroxyapatite crystals at some skeletal sites have a smaller size, with a somewhat reduced total mineral content of the bone. However, these changes are more indicative of increased formation of new bone matrix, which is characterized as having a higher relative content of organic bone matrix. Thus these microscopic observations can be taken as an indication of a potential anabolic effect of strontium treatment on bone turnover.

A significant proof of the skeletal efficacy of strontium treatment comes from the clinical studies of strontium ranelate, which has recently been concluded with two large fracture prevention phase III studies comprising more than 7000 individuals. In the Strontium treated group, 139 patients sustained a new vertebral fracture vs 222 in the placebo group (RR=0.59, 95% Cl=0.48-0.73, P<0.001). The bone formation marker BSAP increased, whereas serum cross-linked C terminal telopeptides of type I collagen (CTX, a specific marker of bone resorption) decreased confirming the potential of strontium ranelate intervention to uncouple the processes of bone formation and resorption (P. J Meunier et al., N EngI J Med, 2004; 350: 459-468).

Accordingly, the present invention relates the use of strontium salts synthesized by the methods described herein, in particular the salts strontium malonate sesquihydrate, strontium di-L-ascorbate di-hydrate, strontium fumarate, strontium salicylate mono-hydrate, strontium succinate and strontium di-ibuprofenate di-hydrate and strontium maleate, for the manufacture of a medicament for the treatment and/or prophylaxis of a cartilage and/or bone disease and/or conditions resulting in a dysregulation of cartilage and/or bone metabolism in a mammal, such as, e.g., a human female or male adult, adolescent or a child, such as, e.g., osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis and for the improvement of fracture healing after traumatic or atraumatic fracture.

### FIGURE LEGENDS

FIG. 1. is a graphic presentation of the asymmetric unit of the new crystalline form of strontium salicylate mono-hydrate. 75% probability ellipsoids and the assigned atomic numbering are depicted. H atoms are depicted as circles of arbitrary size. Atoms labelled with an asterix (*) are at the symmetry positions (see Example 2).

FIG. 2 displays a graphic representation of the crystal packing of strontium salicylate mono-hydrate viewed down the a-axis. The Sr eight-coordination is shown as polyhedra. Hydrogen positions are omitted for clarity.

FIG. 3 is a graphic presentation of the asymmetric unit of the new crystalline form of strontium malonate 1½ hydrate. 75% probability ellipsoids and the assigned atomic numbering are depicted. H atoms are depicted as circles of arbitrary size. O5 denotes the oxygen atom of the water molecule shared between two unit cells of the structure. Atoms labelled with an asterix (*) are at the symmetry positions (see Example 3).

FIG. 4. shows a graphic presentation of the crystal packing of strontium malonate 1½ hydrate viewed down the b-axis. The Sr nine-coordination is shown as polyhedra. Hydrogen positions are omitted for clarity.

FIG. 5 is a graphic presentation of the asymmetric unit of the new crystalline form of strontium di-L ascorbate di-hydrate. 75% probability ellipsoids and the assigned atomic numbering are depicted. H atoms are depicted as circles of arbitrary size. Atoms labelled with an asterix (*) are at the symmetry positions (see Example 4)

FIG. 6 provides a representation of the crystal packing of strontium di-L ascorbate di-hydrate viewed down the a-axis. The Sr eight-coordination is shown as polyhedra. C atoms are slightly larger and lighter than the 0 atoms. Hydrogen positions are omitted for clarity

FIG. 7 is a graphic presentation of the symmetric unit-cell of the new crystalline form of strontium di-ibuprofenate di-hydrate. 75% probability ellipsoids and the assigned atomic numbering are depicted. Hydrogen positions are omitted for clarity. Atoms labelled with an asterix (*) are at the symmetry positions (see Example 5)

FIG. 8 depicts the crystal packing of strontium di-ibuprofenate di-hydrate viewed down the a-axis. The Sr eight-coordinations are shown as polyhedra. Hydrogen positions are omitted for clarity.

In the following is given a more detailed description of the preparation of individual salts according to the invention. The details and particulars described above for strontium salts apply mutatis mutandis to the individual strontium salts, whenever relevant, as well as details and particular described below for the individual strontium salts apply mutatis mutandis to the strontium salts in general, whenever relevant. The present invention is not limited to the above-mentioned specific examples of suitable salts that merely serve as an example of the general applicability of the methods according to the invention. Accordingly, other divalent strontium ion salts containing any of the molecules listed in the preceding sections may be prepared by the manufacturing methods disclosed herein.

### EXAMPALES

### Example 1

### General Method for Preparation of Crystalline Salts by Neutralisation of Carboxylic Acids with Strontium Carbonate Under Room Temperature Conditions

The need for improvement in the known methods for synthesis of metal organic salts of alkaline earth metals is obvious from the Comparison Examples 8 and 9 below. In the present example a new synthesis method is described that allows easy synthesis of pure crystalline forms of metal-organic compounds with temperature sensitive organic anions.

In general the synthesis method can be carried out in laboratory scale as described below:

A small amount of the organic acid proper (0.75-3 g, see Table 1 below) was dissolved in water by heating to temperatures up to 30° C. After cooling to temperatures below 30° C., powdered strontium carbonate (Sigma Aldrich, SrCO₃, MW 147.6, CAS no. 1633-05-02, approx. 10 g/L) was slowly sprinkled over the solution under vigorous stirring by a magnetic stirring rod. Large amounts of carbon dioxide was liberated during the initial steps of adding strontium carbonate, while only traces of gas evolutions was recognized during the final stages of reaction. The majority of salts precipitated in high yield after addition of equimolar amounts of carboxylic acids and strontium carbonate and the precipitate was recovered by filtering (Frisenette 643-111) at room temperature. Small volumes of the filtrate were transferred to beakers where the salts crystallized to larger crystals within 1 to 4 hours. By equimolarity is meant that the amount of negative charges of the anion and positive charges of strontium should approximate a 1:1 relationship, such that a monoprotic acid should be used in an approximate 2:1 relationship and a diprotic acid in an approximate 1:1 relationship to strontium.

Recrystallisation of the precipitated forms did contrary to a priori expectations result in significant reductions in both yield and purity of the precipitated salts. The origin of this behavior of strontium salts may be related to changes or heterogeneity in amounts of crystal water bound but also related to precipitation of strontium carbonate by reaction of strontium ions with carbon dioxide upon cooling of saturated solutions. It also demonstrates the importance of the new method described in this patent as it allows the production of the salts in pure form without the need for subsequent recrystallization, as is used in the conventional prior art methods.

Table 1 below gives an overview of the reaction products and resulting salts obtained when employing the method according to the present invention for manufacture of strontium salts of heat labile and/or pH sensitive carboxylic acid anions.

TABLE 1
Table 1. Conditions and results of the synthesis of strontium salts by reaction of
strontium carbonate with the anion dissolved in water. Crystal structures and
diffractograms were obtained as described in Example 7. The structures were resolved
by single-crystal x-ray crystallography and the results were compared to data of the
Cambridge Crystallographic Database, which unambiguously identified the new
compound.

| STRONTIUM | | | | | | | |
|---|---|---|---|---|---|---|---|
| SALT | | | | | | | |
| OF (FREE | MW | | | FREE | AMOUNT | | |
| ACID | (g/mol) | | SrCO₃ | ACID | OBTAINED | POWDER | CRYSTAL |
| USED): | acid | CAS | (g used) | (g used) | (g) | X-RAY | STRUCTURE |
| Fumaric | 116.08 | 110-17-8 | 29.53 | 23.22 | 37.00 | Yes | Yes |
| acid | | | (0.20 mol) | (0.20 mol) | | | |
| Salicylic | 138.12 | 69-72-7 | 50.00 | 47.00 | 80.00 | Yes | Yes |
| acid | | | (0.34 mol) | (034 mol) | | | |
| Succinic | 118.09 | 110-15-6 | 29.53 | 23.60 | 35.63 | Yes | No |
| acid | | | (0.20 mol) | (0.20 mol) | | | |
| Malonic | 104.06 | 141-82-2 | 14.80 | 10.40 | 17.28 | Yes | Yes |
| acid | | | (0.1 mol) | (0.1 mol) | | | |

### Example 2

### Synthesis of Strontium 2-oxido-Bensoate Monohydrate (Strontium Salicylate)

Strontium 2-oxido-bensoate hydrate was synthesized according to the method described in example 1. Briefly described, strontium carbonate was added in equimolar amounts to a saturated solution of salicylic acid at 40° C. The saturated solution was prepared by dissolving 47 g of salicylic acid (Sigma S5922, MW 138.12) in 250 ml of deaerated distilled water. After complete dissolution of solid salicylate, 50 g of strontium carbonate (Sigma Aldrich, SrCO₃, MW 147.6, CAS no.1633-05-02) was added under constant mixing over a time period of approximately 30 minutes. Strontium 2-oxido-bensoate hydrate was obtained in a yield of more than 95% of the theoretical amount and high purity by precipitation at 20° C.

This new strontium salt differs substantially from strontium di-salicylate dihydrate which has been described previously (Debuyst et al. 1979, J. Chim. Phys. Chim. Biol. 76, 1117) which has two salicylate atoms pr. Strontium atom, as only the carboxyl group of the salicylate is deprotonated. This gives a much lower molar ratio of strontium atom pr unit weight and thus being less suitable for pharmaceutical applications. Furthermore, the yield and purity reported by Debuyst et al is substantially lower than what we obtain with the new method described here.

The synthesis method disclosed in the present example allowed the production of pure homogeneous single crystals of strontium salicylate monohydrate. The crystal structure was determined by X-ray crystallography as described in example 7.

The crystal data for strontium 2-oxido-bensoate hydrate (strontium salicylate) are as follows:

| | |
|---|---|
| Sr.C₇H₄O₃•H₂O | Mo Kα radiation |
| Mr = 241.74 | λ = 0.71073 Å |
| Mono clinic P2₁/n | Cell parameters from 4077 reflections |
| a = 5.0993 (4) Å | θ = 3.23-30.40° |
| b = 22.808 (2) Å | μ = 7.022 mm⁻¹ |
| c = 6.9811 (6) Å | T = 120(2) K |
| β = 109.755 (2) Å | irregular |
| V = 764.15 (11) Å³ | Colorless |
| Z = 4 | 0.14 × 0.10 × 0.02 mm |
| Dₓ = 2.101 Mg m⁻³ | |
| Dₘ not measured | |
| Data collection | |
| Bruker SMART APEX diffractometer | 10002 measured reflections, |
| | 2251 independent reflections |
| Omega scan, frame data integration | 1917 reflections with >2σ(I) |
| Absorption correction: | Rᵢₙₜ = 0.0398 |
| multiscan Sheldrick GM (2002), | θₘₐₓ = 30.85° |
| SADABS, Version 2.03, | h = −7 → 7 |
| University of Göttingen, Germany | k = −32 → 32 |
| Please give reference | l = −9 → 10 |
| Tₘᵢₙ = 0.4398, Tₘₐₓ = 0.8723 | every 0 reflections frequency: 0 min intensity |
| | decay: none |
| Refinement for strontium 2-oxido- | |
| bensoate hydrate | |
| Refinement on F² | w = 1/[σ²(Fₒ²) + (0.0573P)² + 0.1094P] |
| R[F² > 2σ(F²)] = 0.0376 | where P = (Fₒ² + 2F_{c}²)/3 |
| wR(F²) = 0.0920 | |
| S = 1.077 | (Δ/σ)ₘₐₓ = 0.000 |
| 2251 reflections | Δρₘₐₓ = 1.871 e Å⁻³ |
| 115 parameters | Δρₘᵢₙ = 1.003 e Å⁻³ |
| H atoms treated by a mixture of independent | Extinction correction: none |
| and constrained refinement | Scattering factors from International |
| | Tables for Crystallography (Vol. C) |

Selected geometric parameters (Å, °) for strontium 2-oxido-bensoate hydrate

| | | | |
|---|---|---|---|
| Sr1—O1 | 2.469 (2) | Sr1—O3ⁱⁱ | 2.605 (2) |
| Sr1—O4 | 2.502 (2) | Sr1—O2ⁱⁱⁱ | 2.666 (2) |
| Sr1—O3ⁱ | 2.579 (2) | Sr1—O1ⁱⁱⁱ | 2.677 (2) |
| Sr1—O2ⁱ | 2.591 (2) | Sr1—O2ⁱⁱ | 2.738 (2) |

Symmetry codes:
ⁱx, y, 1 + z;
ⁱⁱ1 − x, − y, 1 − z;
ⁱⁱⁱ− x, − y, 1 − z.

Hydrogen-bonding geometry (Å, °) for strontium 2-oxido-bensoate hydrate

| D-H . . . A | D-H | H . . . A | D . . . A | D-H . . . A |
|---|---|---|---|---|
| O4—H8 . . . O3ⁱ | 0.823 (18) | 1.90 (2) | 2.718 (3) | 170 (4) |

Symmetry codes:
ⁱx − 1, y, 1 + z.

All H parameters were initially refined freely. In the final cycles the H atoms of the CH group were placed in calculated positions with C—H═0.93 Å, and refined as riding atoms. For the water molecule the O—H distances were restrained to 0.82 (2) Å. The displacement parameters were set to 1.2 (CH) or 1.5 (OH) times Ueq of the corresponding C or O atoms.

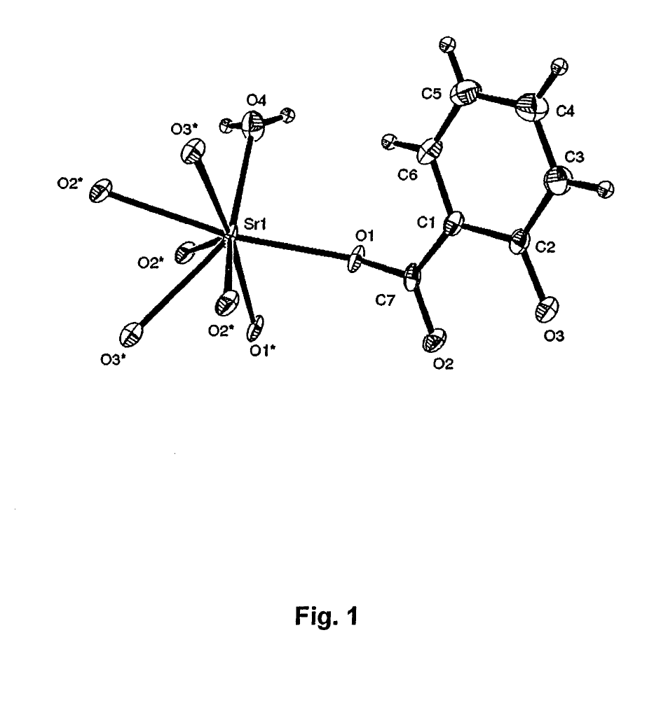

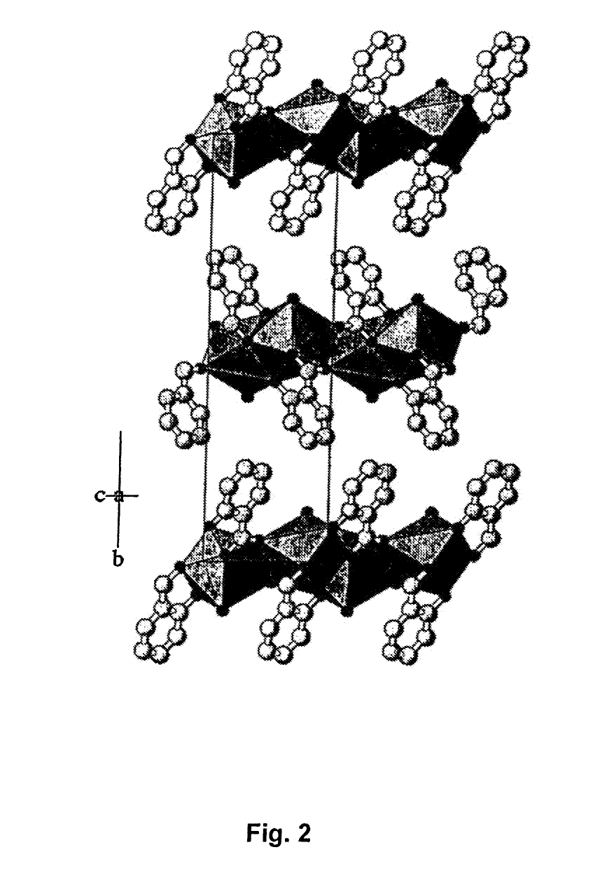

Sr is eight-coordinated in an approximate square antiprisms. The antiprisms are pairwise connected through face-sharing, and these pairs are further connected by edge-sharing into layers in the ac-plane (see FIG. 1). The 2-oxido-bensoates are protruding from the layers and connecting them through van der Waals forces in the b-direction. FIG. 2 shows the crystal packing of strontium salicylate, with strontium shown as eight-coordinated polyhedra.

By comparison, the Sr disalicylate dihydrate (Debyust et al. 1979), form polyhedral chains, where the hydroxyl group takes part in a three-dimensional hydrogen bonding network connecting these chains. In Sr-2-oxido-bensoate hydrate only one of the water H donors, H8, participates in regular hydrogen bonding. The other, H7, does not take part in a conventional hydrogen bond, but points towards the center of a neighbouring benzene ring with a distance of 2.83 Å to the center (A) and an O4-H7-A angle of 154°.

TABLE 2
Fractional atomic coordinates and equivalent isotropic displacement
parameters (Å²) for strontium 2-oxido-bensoate (strontium salicylate,
monohydrate) Ueq = (⅓)ΣᵢΣⱼU^{ij}αⁱα^{j}aᵢ.aⱼ.

| | x | | y | | z | | Ueq | |
|---|---|---|---|---|---|---|---|---|
| Sr1 | 0.21082 | (5) | 0.023582 | (11) | 0.79827 | (4) | 0.00859 | (10) |
| O1 | 0.1711 | (4) | 0.04870 | (9) | 0.4455 | (3) | 0.0124 | (4) |
| O2 | 0.2722 | (4) | 0.01515 | (9) | 0.1812 | (3) | 0.0116 | (4) |
| O3 | 0.6267 | (4) | 0.07957 | (8) | 0.0468 | (3) | 0.0110 | (4) |
| C1 | 0.4460 | (6) | 0.11029 | (12) | 0.3122 | (4) | 0.0101 | (5) |
| C2 | 0.6007 | (6) | 0.11929 | (12) | 0.1780 | (4) | 0.0103 | (5) |
| C3 | 0.7369 | (6) | 0.17410 | (13) | 0.1914 | (5) | 0.0156 | (6) |
| H3 | 0.8377 | | 0.1814 | | 0.1050 | | 0.019 | |
| C4 | 0.7254 | (7) | 0.21696 | (14) | 0.3274 | (5) | 0.0192 | (7) |
| H4 | 0.8188 | | 0.2522 | | 0.3320 | | 0.023 | |
| C5 | 0.5744 | (7) | 0.20779 | (13) | 0.4587 | (5) | 0.0162 | (6) |
| H5 | 0.5679 | | 0.2365 | | 0.5517 | | 0.019 | |
| C6 | 0.4350 | (6) | 0.15527 | (13) | 0.4476 | (5) | 0.0134 | (6) |
| H6 | 0.3304 | | 0.1494 | | 0.5323 | | 0.016 | |
| C7 | 0.2914 | (6) | 0.05545 | (13) | 0.3132 | (4) | 0.0107 | (5) |
| O4 | −0.0506 | (5) | 0.11408 | (9) | 0.8271 | (4) | 0.0169 | (4) |
| H7 | −0.148 | (7) | 0.1349 | (16) | 0.734 | (5) | 0.025 | |
| H8 | −0.164 | (7) | 0.1041 | (17) | 0.881 | (6) | 0.025 | |

### Example 3

### Synthesis of Strontium Malonate 1½ Hydrate and Determination of Crystal Structure and Physiochemical Properties

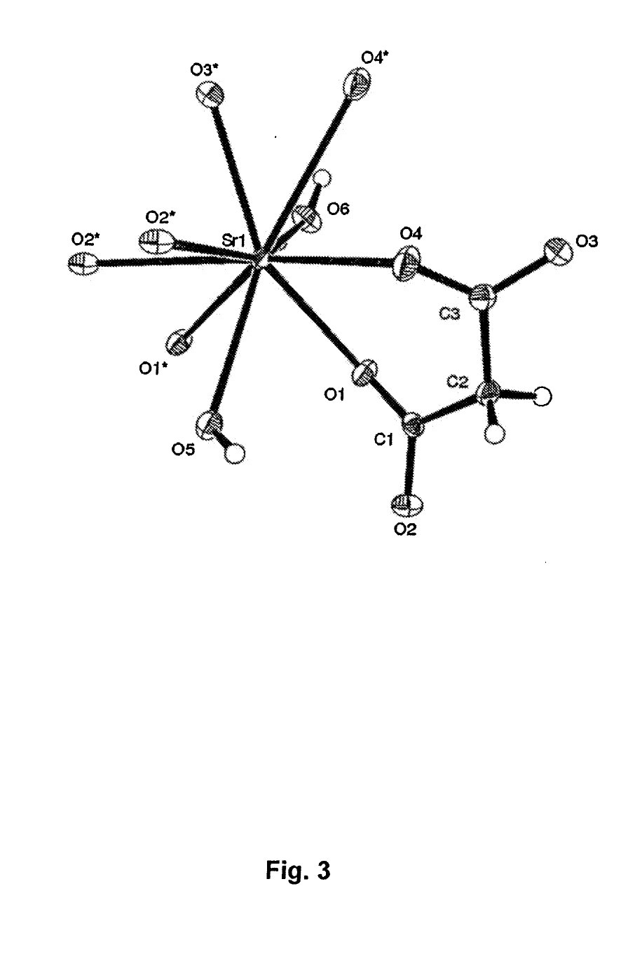

41.6 g of malonic acid (Fluka, MW 104.06 g/mole, CAS no.141-82-2, lot. no. 449503/1, filling code 44903076) was dissolved in water by heating to temperatures up to 30° C. After cooling to temperatures below 30° C., powdered strontium carbonate (Sigma Aldrich, SrCO₃, MW 147.6, CAS no. 1633-05-02,) was slowly sprinkled over the solution under vigorous stirring by a magnetic stirring rod. A total amount of strontium carbonate of 59.05 g was used. During the reaction, large amounts of carbon dioxide was liberated during the initial steps of adding strontium carbonate, while only traces of gas evolutions was observed during the final stages of reaction. The temperature was maintained below 30° C. Strontium malonate 1½ hydrate precipitated as white medium coarse crystals after 60 min of reaction time. The precipitate was recovered by filtering (Frisenette 643-111) at room temperature. The crystal structure of the salt was determined as described in Example 7, and found to have the structure depicted in FIG. 3. Total yield of the salt was 68.5 g and purity was estimated to better than 98%.

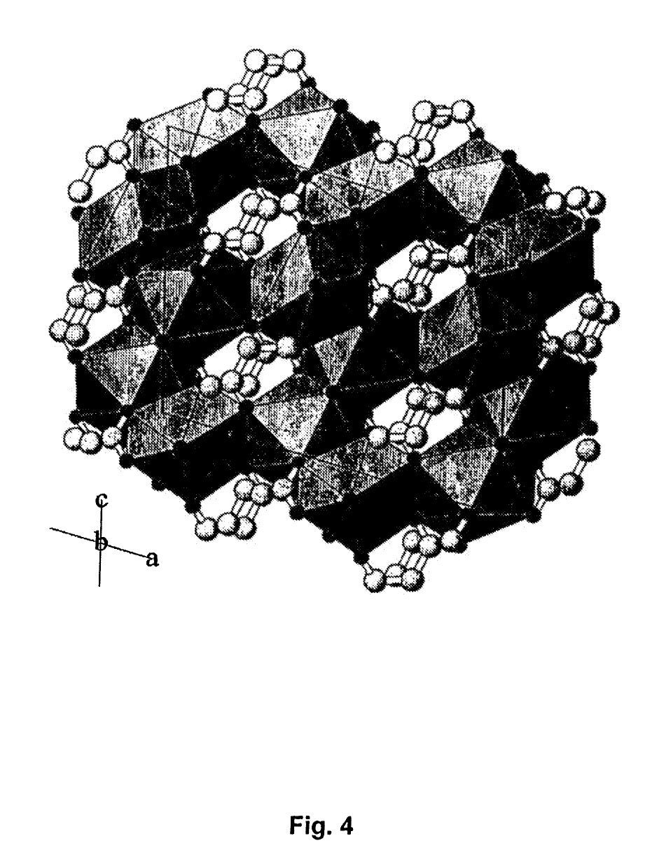

FIG. 4 shows the crystal packing of strontium malonate sesquihydrate, with strontium shown as nine-coordinated polyhedra.

Strontium malonate sesquihydrate was heated to see if the bound crystal water could be removed. The crystal water was irreversibly detached from the malonate at temperatures above approx. 70° C. Thus, anhydrous strontium malonate was produced in high yield and high purity by boiling a solution of strontium malonate. Most likely, both yield and purity could be improved by heating the strontium malonate crystals to even higher temperatures and pressures by applying e.g. an autoclave vessel reaching temperatures of 130° C. and pressures of 2 bar (see patent application PCT/DK2005/000307)

The crystal data for strontium malonate 1½ hydrate (also denoted strontium malonate sesquihydrate) are as follows:

| | |
|---|---|
| 2Sr•2(C₃H₂O₄)•3(H₂O) | Mo Kα radiation |
| Mr = 433.38 | λ = 0.71073 Å |
| Mono clinic C2/c | Cell parameters from 5770 reflections |
| a = 14.3345 (9) Å | θ = 2.97-30.86° |
| b = 7.3458 (5) Å | μ = 9.248 mm⁻¹ |
| c = 11.5075 (7) Å | T = 120(2) K |
| β = 106.7100 (10) Å | irregular |
| V = 1160.55 (13) Å³ | Colorless |
| Z = 4 | 0.33 × 0.30 × 0.08 mm |
| Dₓ = 2.480 Mg m⁻³ | |
| Dₘ not measured | |
| Data collection | |
| Bruker SMART APEX diffractometer | 7363 measured reflections, |
| | 1708 independent reflections |
| Omega scan, frame data Integration | 1630 reflections with >2σ(I) |
| Absorption correction: | Rᵢₙₜ = 0.0228 |
| multiscan Sheldrick GM (2002), | θₘₐₓ = 30.72° |
| SADABS, Version 2.03, | h = −19 → 19 |
| University of Göttingen, Germany | k = −10 → 10 |
| Please give reference | l = −16 → 15 |
| Tₘᵢₙ = 0.06, Tₘₐₓ = 0.48 | every 0 reflections frequency: 0 min intensity |
| | decay: none |
| Refinement for strontium malonate | |
| sesquihydrate | |
| Refinement on F² | w = 1/[σ²(Fₒ²) + (0.0238P)² + 0.6829P] |
| R[F² > 2σ(F²)] = 0.0158 | where P = (Fₒ² + 2F_{c}²)/3 |
| wR(F²) = 0.0413 | |
| S = 1.076 | (Δ/σ)ₘₐₓ = 0.003 |
| 1708 reflections | Δρₘₐₓ = 0.545 e Å⁻³ |
| 97 parameters | Δρₘᵢₙ = −0.485 e Å⁻³ |
| H atoms treated by a mixture of independent | Extinction coefficient: 0.0044 (2) |
| and constrained refinement | Scattering factors from International Tables |
| | for Crystallography (Vol. C) |

Selected geometric parameters (Å, °) for strontium malonate sesquihydrate

| | | | |
|---|---|---|---|
| Sr1—O4 | 2.5386 (10) | Sr1—O1ⁱⁱⁱ | 2.6850 (10) |
| Sr1—O1 | 2.5801 (9) | Sr1—O5 | 2.6956 (9) |
| Sr1—O6 | 2.5839 (10) | Sr1—O2ⁱⁱⁱ | 2.8423 (10) |
| Sr1—O3ⁱ | 2.5942 (9) | Sr1—O4ⁱ | 2.9836 (11) |
| Sr1—O2ⁱⁱ | 2.6201 (10) | | |

Symmetry codes:
ⁱ⅔ − X, ½ − y, 1 − z;
ⁱⁱx, −y, ½ + z;
ⁱⁱⁱ1 − x, y, ½ − z.

Hydrogen-bonding geometry (Å, °) for strontium malonate sesquihydrate

| D-H . . . A | D-H | H . . . A | D . . . A | D-H . . . A |
|---|---|---|---|---|
| O5—H3 . . . O3ⁱ | 0.824 (14) | 1.902 (14) | 2.7165 (12) | 169.7 (19) |
| O6—H4 . . . O3ⁱⁱ | 0.797 (15) | 2.179 (17) | 2.8662 (14) | 144.6 (19) |
| O6—H5 . . . O2ⁱⁱⁱ | 0.805 (15) | 2.150 (16) | 2.9328 (14) | 164.2 (19) |

Symmetry codes:
ⁱ 3/2 − x, y − ½, ½ − z;
ⁱⁱx − ½, ½ + y, z;
ⁱⁱⁱx, 1 − y, ½ + z.

All H parameters were initially refined freely. In the final cycles the H atoms of the CH₂ group were placed in calculated positions with C—H═0.97 Å, and refined as riding atoms. For the water molecules the O—H distances were restrained to 0.82 (2) Å. The displacement parameters were set to 1.2 (CH₂) or 1.5 (OH) times Ueq of the corresponding C or O atoms.

Sr is nine-coordinated by all available malonate and water 0 atoms. The polyhedra are connected by edge and face sharing into a three-dimensional network. O3 and O6 are still unshared between polyhedra. The zeolitelike channel system thus created is occupied by the malonate carbon backbone (FIG. 4). All water H atoms are involved hydrogen bonding to carboxylic O atoms. By comparison, Sr malonate anhydrate (Briggman & Oskarson, 1977) form a similar three-dimensional polyhedral network, but all O atoms are shared between Sr polyhedra. It results in a relatively dense packing, Dₓ=2.78 Mgm⁻³ as compared to 2.48 Mgm⁻³ in strontium malonate sesquihydrate. The higher degree of interconnections and denser packing is the most probable cause of the irreversible dehydration of strontium malonate sesquihydrate.

TABLE 3
Fractional atomic coordinates and equivalent isotropic displacement
parameters (Å²) for strontium malonate sesquihydrate
Ueq = (⅓)ΣᵢΣⱼU^{ij}αⁱα^{j}aᵢ.aⱼ.

| | x | | y | | z | | Ueq | |
|---|---|---|---|---|---|---|---|---|
| Sr1 | 0.587276 | (8) | 0.183425 | (15) | 0.420747 | (9) | 0.00682 | (6) |
| O5 | ½ | | −0.06214 | (19) | ¼ | | 0.0107 | (2) |
| H3 | 0.5347 | (14) | −0.132 | (2) | 0.2248 | (18) | 0.016 | |
| O6 | 0.55766 | (7) | 0.53002 | (14) | 0.39529 | (9) | 0.01319 | (19) |
| H4 | 0.5016 | (11) | 0.551 | (3) | 0.3629 | (17) | 0.020 | |
| H5 | 0.5791 | (14) | 0.617 | (3) | 0.4375 | (17) | 0.020 | |
| O1 | 0.59628 | (7) | 0.26538 | (14) | 0.20622 | (8) | 0.01000 | (18) |
| O2 | 0.59520 | (7) | 0.12878 | (14) | 0.03327 | (8) | 0.01064 | (18) |
| O3 | 0.88290 | (7) | 0.24146 | (14) | 0.35119 | (8) | 0.01177 | (18) |
| O4 | 0.75451 | (7) | 0.12301 | (15) | 0.39481 | (9) | 0.01383 | (19) |
| C1 | 0.63950 | (10) | 0.18388 | (15) | 0.13999 | (12) | 0.0077 | (2) |
| C2 | 0.74886 | (9) | 0.15711 | (18) | 0.18314 | (12) | 0.0099 | (2) |
| H1 | 0.7785 | | 0.2449 | | 0.1417 | | 0.012 | |
| H2 | 0.7634 | | 0.0369 | | 0.1581 | | 0.012 | |
| C3 | 0.79744 | (10) | 0.17521 | (16) | 0.31886 | (12) | 0.0089 | (2) |

### Example 4

### Synthesis of Strontium Di L-Ascorbate Dihydrate

Formation of single crystals of strontium di L-ascorbate dihydrate was performed in line with the method described by S. L. Ruskin and A. T. Merrill (Science, May, 1947, p. 504) for producing calcium ascorbate. The method described by Ruskin and Merrill provides for production of the calcium salt of L-ascorbic acid at a temperature of 30° C., but requires precipitation of the salt in excess acetone, and results in an amorphous precipitate requiring extensive washing with alcohol/acetone and recrystallization to obtain a homogeneous well defined crystalline form. Furthermore, in the method according to Ruskin & Merrill a molar excess of calcium is used, and product analysis indicate a low yield and purity. We were able to produce strontium L-ascorbate by a method comparable to the method disclosed by Ruskin and Merrill. In short 33.6 g of strontium carbonate (0.22 mol) was added slowly over 1-2 hours to a solution of 40 g dissolved ascorbic acid (0.22 mol). The solution was decanted into a large beaker containing 2.5 L of acetone, which resulted in immediate precipitation of a white compound. This compound was filtered and in the filter, coarse-grained strontium ascorbate was obtained. Crystals suitable for single crystal analysis were obtained after drying in vacuum in a desiccator.

However, the total yield of strontium di L-ascorbate dihydrate obtained by the above mentioned method was rather poor and a recrystallization step was required to obtain sufficient purity and homogeneity of the salt. This is in accordance with the report by Ruskin & Merrill, where a similar recrystallization was required for obtaining calcium L-ascorbate in pure form.

The present inventors have found out that by using an 1:2 molar ratio between strontium carbonate and ascorbic acid, strontium di L-ascorbate dihydrate can be obtained in a yield close to 100%. This corresponds to an equimolar ratio between anion and cation charges. In a title experiment, 16.8 g of strontium carbonate (containing 0.11 mol strontium) was reacted with 40 g of L-ascorbic acid (0.22 mol) in a total volume of 200 mL. A small amount of acetone was added to the solution to induce crystal formation and the solution was filtered and allowed to rest at room temperature (22-24° C.) until significant amounts of crystallized precipitate of the organic strontium salt appeared in the filtrate.

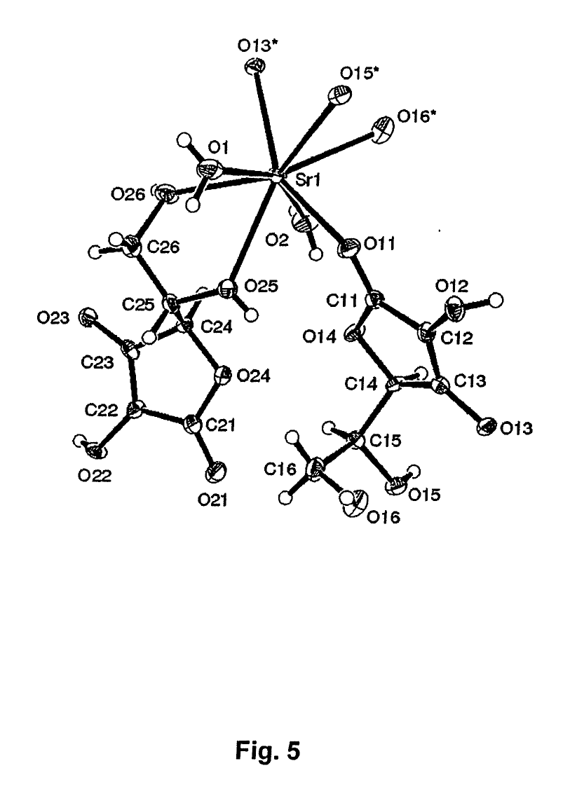

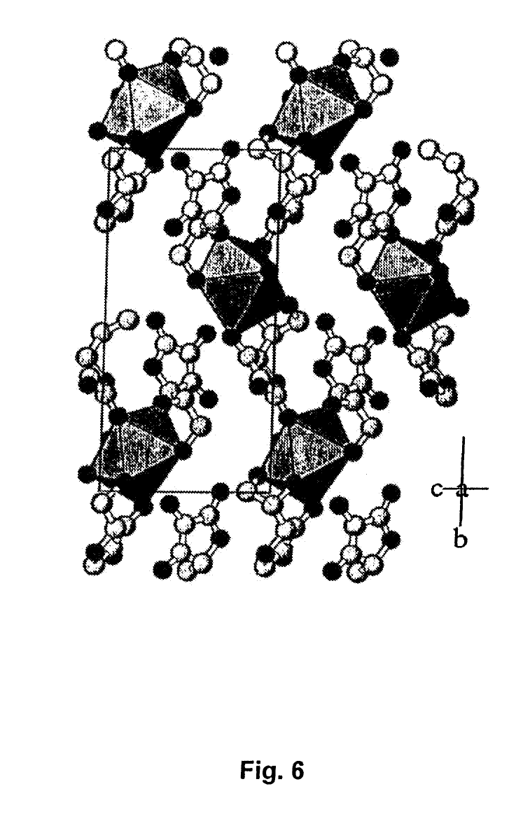

This strontium salt is highly soluble in water and has a pronounced tendency to form a yellow syrup of the compound containing only small amounts of water. Upon drying in vacuum in a desiccator, remaining traces of water is evaporated thus forming white crystalline powder. The crystal structure of the salt was determined as described in Example 7. The structure of the salt is shown in FIG. 5 and the crystal packing in FIG. 6.

The solubility of strontium di L-ascorbate dihydrate exceeded 500 g/l, and thus this strontium salt is likely to be the most highly soluble strontium salt known to man, which may provide some benefits i.e. for pharmaceutical use of the compound. The crystal data for strontium di L-ascorbate dihydrate are as follows:

| | |
|---|---|
| 2(C₆H₇O₆)•2(H₂O)•Sr | Mo Kα radiation |
| Mr = 473.88 | λ = 0.71073 Å |
| Mono clinic P2₁ | Cell parameters from 6673 reflections |
| a = 6.4358 (5) Å | θ = 2.53-30.69° |
| b = 16.1040 (13) Å | μ = 3.343 mm⁻¹ |
| c = 8.3646 (7) Å | T = 120(2) K |
| β = 107.6960 (10) Å | irregular |
| V = 825.90 (12) Å³ | Colorless |
| Z = 2 | 0.28 × 0.05 × 0.04 mm |
| Dₓ = 1.906 Mg m⁻³ | |
| Dₘ not measured | |
| Data collection | |
| Bruker SMART APEX diffractometer | 10978 measured reflections, |
| | 4728 independent reflections |
| Omega scan, frame data Integration | 4507 reflections with >2σ(I) |
| Absorption correction: | Rᵢₙₜ = 0.0231 |
| multiscan Sheldrick GM (2002), | θₘₐₓ = 30.93° |
| SADABS, Version 2.03, | h = −9 → 9 |
| University of Göttingen, Germany | k = −22 → 23 |
| Please give reference | l = −12 → 11 |
| Tₘᵢₙ = 0.4546, Tₘₐₓ = 0.8779 | every 0 reflections frequency: 0 min intensity |
| | decay: none |
| Refinement for strontium di | |
| L-ascorbate dihydrate | |
| Refinement on F² | w = 1/[σ²(Fₒ²) + (0.0321P)² + 0.0000P] |
| R[F² > 2σ(F²)] = 0.0253 | where P = (Fₒ² + 2F_{c}²)/3 |
| wR(F²) = 0.0573 | |
| S = 1.043 | (Δ/σ)ₘₐₓ = 0.000 |
| 4728 reflections | Δρₘₐₓ = 0.726 e Å⁻³ |
| 274 parameters | Δρₘᵢₙ = −0.281 e Å⁻³ |
| H atoms treated by a mixture of independent | Extinction correction: none |
| and constrained refinement | Scattering factors from International Tables |
| | for Crystallography (Vol. C) |

Selected geometric parameters (Å, °) for strontium di L-ascorbate dihydrate

| | | | |
|---|---|---|---|
| Sr1—O11 | 2.5446 (16) | C12—C13 | 1.376 (3) |
| Sr1—O13i | 2.5688 (15) | C13—C14 | 1.528 (3) |
| Sr1—O16i | 2.5699 (16) | C14—C15 | 1.533 (3) |
| Sr1—O2 | 2.5790 (17) | C15—C16 | 1.525 (3) |
| Sr1—O1 | 2.6016 (16) | O21—C21 | 1.239 (3) |
| Sr1—O26 | 2.6138 (15) | O22—C22 | 1.372 (3) |
| Sr1—O25 | 2.6215 (16) | O23—C23 | 1.277 (3) |
| Sr1—O15i | 2.6423 (16) | O24—C21 | 1.365 (3) |
| O11—C11 | 1.232 (3) | O24—C24 | 1.456 (3) |
| O12—C12 | 1.373 (3) | O25—C25 | 1.442 (3) |
| O13—C13 | 1.302 (2) | O26—C26 | 1.430 (3) |
| O14—C11 | 1.383 (3) | C21—C22 | 1.432 (3) |
| O14—C14 | 1.453 (2) | C22—C23 | 1.375 (3) |
| O15—C15 | 1.445 (2) | C23—C24 | 1.525 (3) |
| O16—C16 | 1.437 (3) | C24—C25 | 1.547 (3) |
| C11—C12 | 1.427 (3) | C25—C26 | 1.516 (3) |

Symmetry codes:
ⁱ−X, ½ + y, 2 − z.

Hydrogen-bonding geometry (Å, °) for strontium di L-ascorbate dihydrate

| D-H . . . A | D-H | H . . . A | D . . . A | D-H . . . A |
|---|---|---|---|---|
| O1—H1 . . . O23i | 0.829 (18) | 1.879 (18) | 2.708 (2) | 177 (3) |
| O1—H1 . . . O21ii | 0.797 (17) | 1.963 (19) | 2.736 (2) | 163 (3) |
| O2—H2 . . . O12iii | 0.820 (17) | 2.104 (18) | 2.920 (2) | 175 (3) |
| O2—H2B . . . O15iv | 0.804 (17) | 2.25 (2) | 2.971 (2) | 150 (3) |
| O12—H12 . . . O23v | 0.821 (17) | 1.756 (18) | 2.571 (2) | 172 (3) |
| O15—H15 . . . O22vi | 0.791 (17) | 1.978 (17) | 2.768 (2) | 177 (3) |
| O16—H16 . . . O21i | 0.802 (18) | 1.997 (19) | 2.783 (2) | 167 (3) |
| O22—H22 . . . O13vii | 0.782 (17) | 1.86 (2) | 2.579 (2) | 154 (3) |
| O25—H25 . . . O14 | 0.783 (17) | 2.25 (2) | 2.893 (2) | 140 (3) |
| O26—H26 . . . O1iii | 0.785 (17) | 2.348 (19) | 3.100 (2) | 161 (4) |

Symmetry codes:
ⁱx − 1, y, z;
ⁱⁱ−x, ½ + y, 1 − z;
ⁱⁱⁱ1 + x, y, z;
^{iv}1 − x, ½ + y, 2 − z;
^{v}x − 1, y, 1 + z;
^{vi}x, y, 1 + z;
^{vii}1 + x, y, z − 1.

All H parameters were initially refined freely. In the final cycles the H atoms of the CH₂ and CH groups were placed in calculated positions with C—H═0.97 Å (CH₂) and 0.98 Å (CH), and refined as riding atoms. For the water molecules and OH groups the O—H distances were restrained to 0.82 (2) Å. The displacement parameters were set to 1.2 (CH₂ and CH) or 1.5 (OH) times Ueq of the corresponding C or O atoms.

Sr is eight-coordinated by ascorbate and water O atoms. The two independent ascorbates are coordinated differently: Ascorbate number 1 uses O11, O13, O15 and O16 to coordinate two Sr ions, thus connecting Sr polyhedra into zigzagging chains in the b-direction; while the No. 2 ascorbate has a one-sided coordination through O25 and O26. The polyhedral chains are further connected by hydrogen bonding in the ac-plane. The conformations of the independent ascorbates are also different: The O14-C14-C25-O25 and O24-C24-C25-O25 are 169.7 (2)° and 57.1 (2)° respectively (FIG. 5). All hydrogen donors are involved in hydrogen bonding participating in a three-dimensional network.

As mentioned above FIG. 6 shows the crystal packing of strontium L-ascorbate dihydrate, with strontium shown as eight-coordinated polyhedra.

TABLE 4
Fractional atomic coordinates and equivalent isotropic displacement
parameters (Å²) for strontium di L-ascorbate dihydrate
Ueq = (⅓)ΣiΣjU^{ij}aⁱa^{j}aᵢ.aⱼ.

| | x | | y | | z | | U_{eq} | |
|---|---|---|---|---|---|---|---|---|
| Sr1 | 0.02286 | (3) | 0.889795 | (13) | 0.77741 | (2) | 0.00880 | (5) |
| O1 | −0.3373 | (3) | 0.87790 | (10) | 0.5303 | (2) | 0.0173 | (3) |
| H1 | −0.367 | (5) | 0.8328 | (14) | 0.481 | (4) | 0.026 | |
| H2 | −0.353 | (5) | 0.9149 | (15) | 0.464 | (3) | 0.026 | |
| O2 | 0.4158 | (3) | 0.84918 | (11) | 0.9468 | (2) | 0.0178 | (3) |
| H3 | 0.456 | (5) | 0.8027 | (13) | 0.983 | (4) | 0.027 | |
| H4 | 0.530 | (3) | 0.8693 | (18) | 0.946 | (4) | 0.027 | |
| O11 | −0.1618 | (3) | 0.78079 | (10) | 0.9079 | (2) | 0.0133 | (3) |
| O12 | −0.4119 | (3) | 0.68869 | (10) | 1.08944 | (19) | 0.0119 | (3) |
| H12 | −0.411 | (5) | 0.6975 | (18) | 1.186 | (2) | 0.018 | |
| O13 | −0.0986 | (3) | 0.54439 | (9) | 1.2563 | (2) | 0.0117 | (3) |
| O14 | 0.0906 | (2) | 0.67923 | (9) | 0.98560 | (18) | 0.0109 | (3) |
| O15 | 0.2856 | (3) | 0.47033 | (9) | 1.13780 | (19) | 0.0114 | (3) |
| H15 | 0.381 | (4) | 0.4895 | (18) | 1.212 | (3) | 0.017 | |
| O16 | −0.1178 | (3) | 0.45095 | (11) | 0.9253 | (2) | 0.0146 | (3) |
| H16 | −0.242 | (3) | 0.4591 | (19) | 0.871 | (3) | 0.022 | |
| C11 | −0.1049 | (3) | 0.71517 | (13) | 0.9845 | (3) | 0.0090 | (4) |
| C12 | −0.2095 | (3) | 0.66672 | (13) | 1.0807 | (2) | 0.0094 | (4) |
| C13 | −0.0767 | (3) | 0.60100 | (13) | 1.1513 | (2) | 0.0094 | (4) |
| C14 | 0.1305 | (3) | 0.60872 | (12) | 1.0993 | (3) | 0.0095 | (4) |
| H14 | 0.2499 | | 0.6238 | | 1.1995 | | 0.011 | |
| C15 | 0.2082 | (4) | 0.53544 | (13) | 1.0153 | (3) | 0.0102 | (4) |
| H17 | 0.3302 | | 0.5542 | | 0.9773 | | 0.012 | |
| C16 | 0.0354 | (4) | 0.49712 | (14) | 0.8664 | (3) | 0.0134 | (4) |
| H18 | −0.0401 | | 0.5406 | | 0.7905 | | 0.016 | |
| H19 | 0.1045 | | 0.4606 | | 0.8054 | | 0.016 | |
| O21 | 0.4790 | (2) | 0.49852 | (11) | 0.70847 | (19) | 0.0145 | (3) |
| O22 | 0.6075 | (3) | 0.53606 | (10) | 0.4076 | (2) | 0.0123 | (3) |
| H22 | 0.704 | (4) | 0.5523 | (18) | 0.377 | (4) | 0.018 | |
| O23 | 0.5624 | (3) | 0.72781 | (10) | 0.3809 | (2) | 0.0124 | (3) |
| O24 | 0.4032 | (3) | 0.63437 | (9) | 0.71404 | (18) | 0.0128 | (3) |
| O25 | 0.0576 | (3) | 0.74240 | (10) | 0.6550 | (2) | 0.0130 | (3) |
| H25 | 0.093 | (5) | 0.7074 | (15) | 0.723 | (3) | 0.020 | |
| O26 | 0.1785 | (3) | 0.87959 | (11) | 0.52356 | (19) | 0.0144 | (3) |
| H26 | 0.296 | (3) | 0.891 | (2) | 0.521 | (3) | 0.022 | |
| C21 | 0.4774 | (4) | 0.56818 | (15) | 0.6452 | (3) | 0.0106 | (4) |
| C22 | 0.5420 | (4) | 0.59363 | (14) | 0.5032 | (3) | 0.0094 | (4) |
| C23 | 0.5169 | (4) | 0.67820 | (14) | 0.4846 | (3) | 0.0097 | (4) |
| C24 | 0.4051 | (4) | 0.70724 | (13) | 0.6114 | (3) | 0.0098 | (4) |
| H24 | 0.4840 | | 0.7535 | | 0.6794 | | 0.012 | |
| C25 | 0.1622 | (4) | 0.72909 | (13) | 0.5272 | (3) | 0.0110 | (4) |
| H27 | 0.0912 | | 0.6822 | | 0.4570 | | 0.013 | |
| C26 | 0.1212 | (4) | 0.80711 | (14) | 0.4207 | (3) | 0.0135 | (4) |
| H28 | 0.2067 | | 0.8052 | | 0.3432 | | 0.016 | |
| H29 | −0.0316 | | 0.8098 | | 0.3555 | | 0.016 | |

### Example 5

### Synthesis of Strontium Di-Ibuprofenate Dihydrate

Ibuprofen is a non-steroidal analgesic agent exerting its physiological action through inhibition of cyclo-oxygenases, used in many pharmaceutical products for relief of pain and aches. We synthesized a novel strontium salt of ibuprofen by the method according to example 1. Briefly described, solid strontium carbonate (Sigma Aldrich, SrCO₃, MW 147.6, CAS no. 1633-05-02) (7.38 g) was added to a solution saturated with ibuprofen (Sigma Aldrich 17905, FW 206.28) (22.83 g) in a total volume of 350 ml at 44° C. over a period of approximately 30 min. The product was obtained in high yield and purity after cooling to room temperature (20° C.), filtration and drying at 40° C.

The crystal data for strontium di-ibuprofenate dihydrate was determined by the method described in example 7. The crystal coordinates are as follows:

| | |
|---|---|
| 2(C₁₃H₁₇O₂)•2(H₂O)•Sr | Mo Kα radiation |
| Mr = 534.18 | λ = 0.71073 Å |
| Triclinic P1 | Cell parameters from 3382 reflections |
| a = 7.9116 (7) Å | θ = 2.34-27.58° |
| b = 10.4870 (10) Å | μ = 1.952 mm⁻¹ |
| c = 18.2493 (17) Å | T = 120(2) K |
| α = 86.088 (2) Å | plate |
| β = 79.784 (2) Å | colorless |
| γ = 70.605 (2) Å | 0.35 × 0.06 × 0.03 mm |
| V = 1405.5 (2) Å³ | |
| Z = 2 | |
| Dₓ = 1.262 Mg m⁻³ | |
| Dₘ not measured | |
| Data collection | |
| Bruker SMART APEX diffractometer | 19139 measured reflections, |
| | 8160 independent reflections |
| Omega scan, frame data | 5038 reflections with >2σ(I) |
| integration | |
| Absorption correction: | Rᵢₙₜ = 0.0511 |
| multiscan Sheldrick GM (2002), | θₘₐₓ = 31.01° |
| SADABS, Version 2.03, | h = −11 → 11 |
| University of Göttingen, Germany | k = −15 → 14 |
| Please give reference | l = −26 → 26 |
| Tₘᵢₙ = 0.5482, Tₘₐₓ = 0.9438 | every 0 reflections frequency: 0 min intensity |
| | decay: none |
| Refinement for strontium di-ibuprofenate | |
| dihydrate | |
| Refinement on F² | w = 1/[σ²(Fₒ²) + (0.0724P)² + 0.0000P] |
| R[F² > 2σ(F²)] = 0.0581 | where P = (Fₒ² + 2F_{c}²)/3 |
| wR(F²) = 0.1450 | |
| S = 0.982 | (Δ/σ)ₘₐₓ = 0.001 |
| 8160 reflections | Δρₘₐₓ = 0.793 e Å⁻³ |
| 310 parameters | Δρₘᵢₙ = −0.439 e Å⁻³ |
| H atoms treated by a mixture of independent | Extinction correction: none |
| and constrained refinement | Scattering factors from International Tables |
| | for Crystallography (Vol. C) |

Selected geometric parameters (Å, °) for strontium di-ibuprofenate dihydrate

| | | | |
|---|---|---|---|
| Sr1—O11ⁱ | 2.476 (2) | Sr1—O12 | 2.595 (2) |
| Sr1—O31ⁱⁱ | 2.486 (2) | Sr1—O32 | 2.599 (3) |
| Sr1—O3 | 2.563 (3) | Sr1—O31 | 2.728 (2) |
| Sr1—O4 | 2.563 (3) | Sr1—O11 | 2.742 (2) |

Symmetry codes:
ⁱ1 − x, 1 − y, −z;
ⁱⁱ2 − x, 1 − y, −z.

Hydrogen-bonding geometry (Å, °) for strontium di L-ascorbate dihydrate

| D—H . . . A | D—H | H . . . A | D . . . A | D—H . . . A |
|---|---|---|---|---|
| O3—H31 . . . O12ⁱ | 0.804 (18) | 1.92 (2) | 2.706 (3) | 165 (4) |
| O4—H41 . . . O32ⁱⁱ | 0.798 (19) | 1.91 (2) | 2.704 (3) | 171 (5) |

Symmetry codes:
ⁱ2 − x, 1 − y, −z;
ⁱⁱ1 − x, 1 − y, −z.

Several of the terminal methyl groups show signs of disorder. However, an attempt to refine C19, C22 and C23 with split position did not improve the overall fit. An anisotropic model was therefore judged as appropriate with the present resolution. All H parameters were initially refined freely. In the final cycles the H atoms of the CH, CH₂ and CH₃ and groups were placed in calculated positions with C—H═0.93 Å (aromatic CH), 0.98 Å (aliphatic CH), 0.97 Å (CH₂) and 0.96 Å (CH₃) and refined as riding atoms. For the water molecules the O—H distances were restrained to 0.82 (2) Å. The displacements parameters were set to 1.2 (CH, CH₂ and CH₃) or 1.5 (OH) time U_{eq} of the corresponding C or O atoms.

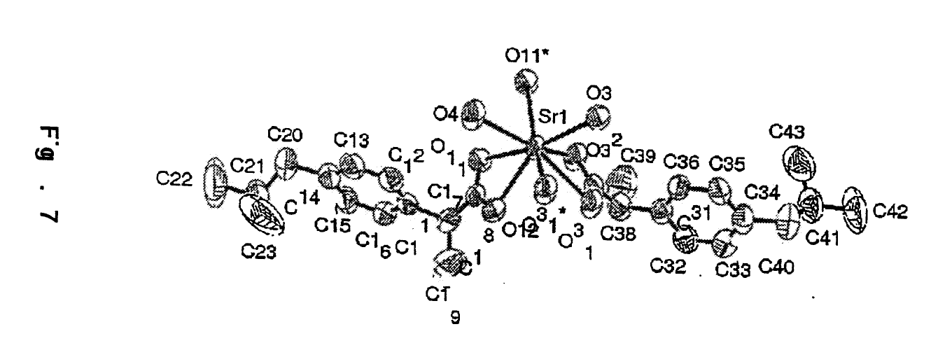

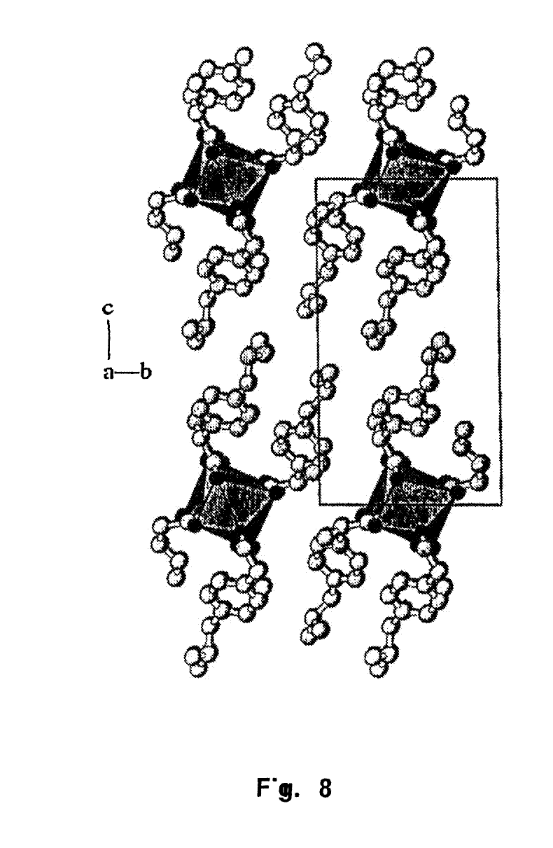

Sr is eight-coordinated in a distorted square antiprism by six O atoms from the asymmetric unit and two additional carboxylate O atoms from neighbouring ibuprofenates (O11 and O31, FIG. 7). The strontium polyhedra share edges to form chains in the a-direction (FIG. 8). The chains are stacked in layers in the ab-plane with the ibuprofenates protruding in the c-direction. These layers are in turn stacked in the c-direction, in both cases by van der Waals interactions only. Viewed in the a-direction (FIG. 8) the strontium polyehedra appear slightly rotated with respect to the ab-plane. This causes a difference in the packing of the two independent ibuprofenates. The one ibuprofenate extends further towards the next layer then the other, which is more confined to the space in between the chains. This difference in packing explains the observation of larger disorder of the terminal methyl groups of the former ibuprofenate. Hydrogen bonding plays a minor role only in the packing. Only one of each water hydrogen is employed in hydrogen bonding, and hydrogen bonding is by the bulkiness of the ibuprofenates restricted to carboxylic O atoms in neighbouring strontium polyhedra within a polyhedral chain.

TABLE 5
Fractional atomic coordinates and equivalent isotropic displacement
parameters (Å²) for strontium di-ibuprofenate dihydrate
Ueq = (⅓)ΣiΣjU^{ij}aⁱa^{j}aᵢ.aⱼ.

| | x | | y | | z | | U_{eq} | |
|---|---|---|---|---|---|---|---|---|
| Sr1 | 0.76760 | (4) | 0.44979 | (4) | 0.012433 | (17) | 0.03868 | (12) |
| O3 | 0.8918 | (3) | 0.3797 | (3) | 0.13485 | (14) | 0.0453 | (6) |
| H31 | 0.976 | (4) | 0.403 | (4) | 0.139 | (2) | 0.068 | |
| H32 | 0.819 | (5) | 0.400 | (4) | 0.1733 | (16) | 0.068 | |
| O4 | 0.7381 | (3) | 0.2360 | (3) | −0.03646 | (15) | 0.0476 | (6) |
| H41 | 0.641 | (4) | 0.253 | (4) | −0.049 | (2) | 0.071 | |
| H42 | 0.815 | (5) | 0.179 | (3) | −0.065 | (2) | 0.071 | |
| O11 | 0.5234 | (3) | 0.5662 | (3) | −0.08197 | (13) | 0.0463 | (6) |
| O12 | 0.8107 | (3) | 0.5425 | (3) | −0.12268 | (13) | 0.0464 | (6) |
| C11 | 0.5004 | (4) | 0.5821 | (4) | −0.24407 | (18) | 0.0370 | (8) |
| C12 | 0.5251 | (4) | 0.4451 | (4) | −0.23561 | (19) | 0.0405 | (8) |
| H12 | 0.5999 | | 0.3940 | | −0.2029 | | 0.049 | |
| C13 | 0.4399 | (5) | 0.3828 | (4) | −0.27514 | (18) | 0.0424 | (8) |
| H13 | 0.4583 | | 0.2907 | | −0.2685 | | 0.051 | |
| C14 | 0.3282 | (5) | 0.4564 | (4) | −0.32417 | (18) | 0.0417 | (8) |
| C15 | 0.3020 | (5) | 0.5935 | (4) | −0.33199 | (19) | 0.0427 | (8) |
| H15 | 0.2263 | | 0.6450 | | −0.3643 | | 0.051 | |
| C16 | 0.3874 | (5) | 0.6554 | (4) | −0.29224 | (18) | 0.0405 | (8) |
| H16 | 0.3678 | | 0.7477 | | −0.2983 | | 0.049 | |
| C17 | 0.6473 | (4) | 0.5823 | (4) | −0.13118 | (19) | 0.0409 | (8) |
| C18 | 0.5944 | (6) | 0.6549 | (4) | −0.2032 | (2) | 0.0534 | (10) |
| H18 | 0.5035 | | 0.7425 | | −0.1884 | | 0.064 | |
| C19 | 0.7462 | (8) | 0.6859 | (7) | −0.2536 | (3) | 0.110 | (2) |
| H19A | 0.7035 | | 0.7314 | | −0.2975 | | 0.132 | |
| H19B | 0.8430 | | 0.6032 | | −0.2673 | | 0.132 | |
| H19C | 0.7899 | | 0.7429 | | −0.2283 | | 0.132 | |
| C20 | 0.2412 | (7) | 0.3893 | (5) | −0.3721 | (2) | 0.0651 | (12) |
| H20A | 0.1134 | | 0.4414 | | −0.3690 | | 0.078 | |
| H20B | ¼ | | 0.2994 | | −0.3526 | | 0.078 | |
| C21 | 0.3331 | (10) | 0.3791 | (6) | −0.4542 | (2) | 0.094 | (2) |
| H21 | 0.3255 | | 0.4706 | | −0.4724 | | 0.113 | |
| C22 | 0.2258 | (13) | 0.3248 | (8) | −0.4996 | (3) | 0.177 | (5) |
| H22A | 0.1013 | | 0.3823 | | −0.4927 | | 0.212 | |
| H22B | 0.2320 | | 0.2348 | | −0.4830 | | 0.212 | |
| H22C | 0.2773 | | 0.3236 | | −0.5514 | | 0.212 | |
| C23 | 0.5376 | (11) | 0.2925 | (6) | −0.4625 | (3) | 0.129 | (3) |
| H23A | 0.5984 | | 0.3328 | | −0.4345 | | 0.155 | |
| H23B | 0.5917 | | 0.2887 | | −0.5141 | | 0.155 | |
| H23C | 0.5488 | | 0.2027 | | −0.4439 | | 0.155 | |
| O31 | 0.8989 | (3) | 0.6557 | (3) | 0.02531 | (13) | 0.0481 | (6) |
| O32 | 0.6074 | (3) | 0.6942 | (3) | 0.06225 | (14) | 0.0490 | (6) |
| C31 | 0.8393 | (4) | 0.8894 | (3) | 0.12206 | (17) | 0.0351 | (7) |
| C32 | 0.8892 | (5) | 1.0051 | (4) | 0.1233 | (2) | 0.0446 | (8) |
| H32A | 0.8533 | | 1.0742 | | 0.0888 | | 0.053 | |
| C33 | 0.9909 | (5) | 1.0190 | (4) | 0.1747 | (2) | 0.0473 | (9) |
| H33 | 1.0230 | | 1.0967 | | 0.1742 | | 0.057 | |
| C34 | 1.0450 | (5) | 0.9188 | (4) | 0.22697 | (19) | 0.0427 | (8) |
| C35 | 0.9992 | (5) | 0.8025 | (3) | 0.22540 | (18) | 0.0390 | (8) |
| H35 | 1.0367 | | 0.7334 | | 0.2596 | | 0.047 | |
| C36 | 0.8985 | (4) | 0.7874 | (3) | 0.17374 | (18) | 0.0367 | (7) |
| H36 | 0.8701 | | 0.7082 | | 0.1736 | | 0.044 | |
| C37 | 0.7435 | (4) | 0.7334 | (4) | 0.05112 | (18) | 0.0414 | (8) |
| C38 | 0.7215 | (5) | 0.8792 | (4) | 0.0677 | (2) | 0.0456 | (9) |
| H38 | 0.7601 | | 0.9207 | | 0.0207 | | 0.055 | |
| C39 | 0.5246 | (6) | 0.9680 | (5) | 0.0984 | (3) | 0.0775 | (14) |
| H39A | 0.4450 | | 0.9646 | | 0.0650 | | 0.093 | |
| H39B | 0.5207 | | 1.0597 | | 0.1026 | | 0.093 | |
| H39C | 0.4862 | | 0.9346 | | 0.1465 | | 0.093 | |
| C40 | 1.1434 | (6) | 0.9389 | (4) | 0.2874 | (2) | 0.0555 | (10) |
| H40A | 1.2437 | | 0.9697 | | 0.2647 | | 0.067 | |
| H40B | 1.1934 | | 0.8530 | | 0.3119 | | 0.067 | |
| C41 | 1.0180 | (7) | 1.0416 | (4) | 0.3455 | (2) | 0.0614 | (12) |
| H41A | 0.9715 | | 1.1280 | | 0.3196 | | 0.074 | |
| C42 | 1.1266 | (9) | 1.0630 | (5) | 0.4018 | (3) | 0.097 | (2) |
| H42A | 1.2278 | | 1.0883 | | 0.3762 | | 0.117 | |
| H42B | 1.1703 | | 0.9807 | | 0.4294 | | 0.117 | |
| H42C | 1.0501 | | 1.1335 | | 0.4355 | | 0.117 | |
| C43 | 0.8562 | (7) | 1.0009 | (5) | 0.3824 | (3) | 0.0766 | (14) |
| H43A | 0.7930 | | 0.9869 | | 0.3449 | | 0.092 | |
| H43B | 0.7757 | | 1.0712 | | 0.4152 | | 0.092 | |
| H43C | 0.8973 | | 0.9187 | | 0.4103 | | 0.092 | |

### Example 6

### General Method for Preparation of Crystalline Salts of Strontium by Precipitation from Dissolved Strontium Chloride and Dissolved Sodium Salts of the Appropriate Carboxylic Anions at Room Temperature

In a glass-beaker of volume 200 mL, 0.1 mol of the sodium salt of the carboxylic acid was dissolved in a small volume of water at room temperature. The final volume was 50 mL. In another beaker 0.05 mol SrCl₂ (SrCl₂ hexahydrate, Sigma-Aldrich 43,966-5) was dissolved in 100 mL of water. This latter solution was slowly decanted into the first solution of the dissolved sodium salt, which resulted in formation of a fine-grained white precipitate. The solution was filtered and allowed to rest at room temperature (22-24° C.) for several days until significant amounts of crystallized precipitate of the organic strontium salt appeared in the filtrate. Strontium salts of ibuprofenate and maleate were obtained by this procedure, as seen in Table 6.

Furthermore, in line with the above the present inventors has discovered and developed a novel synthesis method whereby they were able to synthesize strontium L-ascorbate dihydrate without the need for adding acetone. Strontium chloride hexahydrate was added to sodium L-ascorbate resulting in a final molar ratio of 1:2 as follows: strontium chloride (SrCl₂ hexahydrate, Sigma-Aldrich 43,966-5), approximately 100 g in total was added to an aqueous saturated solution containing approximately 71 g sodium L-ascobate (Sigma-Aldrich A7631, MW 198.11). After addition of the strontium chloride more sodium L-ascorbate, approximately 77 g in total, was added to the solution at a temperature of 44° C. until a transparent yellow colored syrup was obtained. The syrup was dried initially by suction filtering followed by drying in a desiccator. The final product thus obtained was a white powder with a yellow tarnish, while the selected single crystals appeared colorless.

TABLE 6
Conditions and results of synthesis of strontium salts by reaction of strontium
chloride with the appropriate sodium salt of the anion. Crystal structures and
diffractograms were obtained as described in Example 7. Analysis of the powder X-ray
crystallographic analysis of the strontium di-ibuprofen salt enabled identification of the
crystal structure of the obtained salt as being identical to the strontium di-ibuprofenate
di-hydrate salt, shown in FIGS. 7 and 8. The salt obtained in the synthesis reaction
with L-ascorbate was identified as strontium di L-ascorbate dihydrate as shown in
FIGS. 5 and 6.

| | MW | | | | | | |
|---|---|---|---|---|---|---|---|
| Strontium salt | (g/mol) | | | | | | |
| of (sodium salt | of | | | Sodium | Amount | | |
| used for | sodium | | SrCL₂•6H₂O | salt | obtained | Powder | Crystal |
| synthesis): | salt | CAS | (g used) | (g used) | (g) | X-ray | struc. |
| Ibuprofenate | 228.29 | 31121- | 13.33 | 22.83 | 26.33 | Yes | No |
| | | 93-4 | | | | | |
| Maleate | 160.04 | 371-47-1 | 12.49 | 15.00 | 14.30 | Yes | No |
| Ascorbate | 198.11 | 134-03-2 | 100 | 148 | | Yes | Yes |

### Example 7

### Determination of Crystal Structure by X-RAY Diffraction

### General

Crystalline material is defined as having a structure with a three-dimensional repetition, i.e. there is a smallest identical unit, the unit cell, which by translations in three dimensions will fit to any part of the crystal. The unit cell dimensions are typically between 3 and 25 Å for inorganic and organic materials. Such a three-dimensional array of unit cells will also contain sets of lattice planes connecting all corners of the unit cells. The distance between the lattice planes in such a set will be from zero up to the maximum dimension of the unit cell itself. The plane distances are thus in the same order of magnitude as the X-ray wavelength used for diffraction, 0.5-2.4 Å. When such a crystal is placed in an X-ray beam it will act as a grating to create a characteristic interference or diffraction pattern The positions of the recorded diffracted radiation will be determined by the lattice plane distances, i.e. the size of the unit cell, while the recorded diffracted intensities are determined by the positions and symmetry of the atoms in the unit cell. For practical purposes it means that a unique crystal structure will produce a unique diffraction pattern that can be used for identification or to determine the crystal structure. There are two general methods commonly used for structure analysis: The single-crystal method and the powder diffraction method.

### Single-Crystal Methods

This method is primarily used to determine the crystal structures of unknown materials. As the name implies just one crystal, typically less than 0.3 mm in size, is used. The crystal is mounted on a single-crystal diffractometer where it can be rotated in independent directions and a complete three-dimensional diffraction pattern can be collected in about ten hours. From the positions of the diffraction spots we can calculate the unit cell dimensions and from the intensity of the spots we can solve the atomic arrangement within the unit cell. The solved structure is unique within the accuracy, typically better than 0.01 Å in inter-atomic distances and the method is also sensitive to the absolute confirmation of the molecules in the structure. With modern diffractometers and software the method is successful to 99% with organic and metal organic compounds.

### Powder Diffraction

A powder sample will ideally contain an infinite amount of micrometer sized crystals in random orientation. When radiated by X-ray each of the crystallites will diffract independently and add its contribution to the diffraction pattern. As a result a powder diffraction pattern will be a one-dimensional projection of the three-dimensional single-crystal pattern. The interpretation of a powder diffraction pattern is much less straightforward than a single-crystal pattern. Depending on unit cell size and symmetry a powder diffraction pattern show various degrees of reflection overlap. Nevertheless, the peak positions are still a function of the unit cell dimensions and the intensities a function of the unit cell contents. A powder diffraction pattern is more or less a fingerprint of the investigated structure, and using a powder diffraction data base and an effective search-match program we can with 10 minutes of data collection and a few minutes analysis safely identify known structures. Powder diffraction has become the workhorse for structural characterization of materials in general. Except for phase identification, the method is commonly used for structure solution, structure refinements and for studies of crystallinity, crystallite size and size distributions, stress/strain etc. Although the method is primarily intended for solid crystalline materials, information from amorphous and fibrous materials and thin films is also readily obtained.

### Powder Diffraction Equipment

Diffractometer: Huber G670 powder diffractometer operating in Guinier (transmission) geometry and equipped with a primary quartz focusing monochromator and an imaging plate detector with an integrated laser/photomultiplier read-out system

X-ray generator: 40 kV and 30 mA.

Radiation: CuKα1 1.54059 Å

Instrument calibration: Intensity and 2θ-scale checked with a Si-standard (NBS) fitted through full pattern Rietveld refinements. Calibrated approximately once a week and after any adjustment of the diffractometer.

Sample holder: Flat plate scotch tape, 10 by 10 mm active area in Scotch tape

Measurement: Range: 2 to 100° in 2θ. Detector is read out in steps of 0.05° in 2θ. Exposure time is between 15 and 120 min depending on scattering power.

Measurement procedure: The samples are ground by an agate mortar and pestle and put on the sample holder on the Scotch tape. The sample holder is mounted on the powder diffractometer mount and the rocking motor is started. In the data collection program the file name is given (typically the sample name) and any other comments or observations are entered. The measuring time is entered and the data collection started. The file name, measuring time and operator is written in the note book. After completed measurement the powder diffraction pattern is printed and signed by the operator. An attempt to identify the sample using the search-match program will usually be made.

### Comparison Example 8

### Use of a Known Method for Preparation of Crystalline Salts of Strontium by Precipitation from Dissolved Strontium Chloride and Dissolved Sodium Salts of the Appropriate Carboxylic Anions

In this example is shown the result of using methods known in the art to produce strontium salts. The yield of salts produced by this method was generally below 60% and often one or more re-crystallizations are required to obtain the crystalline salt in sufficient purity. As an example of the method the following procedure is provided, which describes a general procedure for synthesis of strontium salts with organic anions that can be performed without any prior knowledge regarding the anion. In a glass-beaker of 100 mL volume, 5 g of the sodium salt of the carboxylic acid was dissolved in a small volume of water that was slightly heated at temperatures not greater than 30-50° C. In a title experiment according to this example, sodium fumarate was used (5 g=0.0312 mol), but other anions may be used. The final volume was adjusted to 25-50 mL. In another beaker 10 g (0.0375 mol) of SrCl₂ (SrCl₂ hexahydrate, Sigma-Aldrich 43,966-5) was dissolved in 100 mL of water. This latter solution was slowly decanted into the first solution of the dissolved sodium salt. The transfer continued until an initial cloudiness was observed, which resulted in a total volume of 50-100 mL. The solution was allowed to rest at room temperature (22-24° C.) for several days until significant amounts of crystallized precipitate of the organic strontium salt appeared.

The reaction that proceeds is exemplified by the reaction between strontium ions and sodium fumarate (reaction schemes (a) and (b)):

$$\begin{matrix}NaOOCCHCHCOONa ( s ) + {H}_{2} O ( l ) {->}^{-} OOCCHCHCOOH ( aq ) + 2 {Na}^{+} ( aq ) + {OH}^{-} ( aq ) & ( a ) \\ { }^{-} OOCCHCHCOOH ( aq ) + {Sr}^{2 +} ( aq ) -> Sr ( OOCCHCHCOO ) ( aq ) + {H}^{+} ( aq ) & ( b )\end{matrix}$$

After the precipitation, the solution was filtered on a Buchner funnel using a suction flask and the crystals were flushed in small volumes of ethanol. Crystals of some of the salts were very soluble, so in order to improve the yield of crystals, the solution was allowed to rest longer, such as at least 30-60 min. Repeated crystallization resulted in yields of approximately 50%. Strontium salts of L-aspartate and of lactate were very soluble, with solubility exceeding 25 g/l in water at room temperature.

The lactate and L-glutamate salts of strontium were precipitated from solutions with an excess of strontium chloride and large crystals of the lactate salt were achieved by slow evaporation of the solvent.

### Comparison Example 9

### General Method for Preparation of Crystalline Salts by Neutralization of Carboxylic Acids with Strontium Hydroxide

This example provides another method known in the art to prepare alkaline metal salts of carboxylic acid anions, using the hydroxide salt of strontium as a starting point for the synthesis. A small amount of the appropriate organic acid proper (0.75-3 g, see table below) was dissolved in water by heating to temperatures between 30° C.-50° C. Then, strontium hydroxide (Sigma Aldrich, Sr(OH)₂*8H₂O, MW 265.71, CAS no. 1311-10-0, approx. 10 g/L) was slowly added. Then, a magnetic stirring rod was added and the stirring and gentle heating (i.e. 30-50° C.) of the suspension was started. After some time, the solution clarifies and all the solid material dissolves. The heating is maintained, and after three hours of incubation, the solution is filtered while hot on a Buchner funnel. Very small amounts of impurities were left in the filter.

The filtrate was subsequently allowed to cool at room temperature overnight, which resulted in growth of fine-powdered crystals of the desired strontium salt. Further purifications of the salts can be performed by repeated re-crystallizations (Table 7).

TABLE 7
Amounts of start reagent used for organic strontium salt synthesis and
recoveries in the synthesis of eight specific organic strontium salts following the
general reaction pathway with free-acid forms of the anion, and strontium hydroxide

| Strontium salt | | | | | | |
|---|---|---|---|---|---|---|
| of (free acid | | | Amount | Estimated | | Crystal |
| used): | Sr(OH)₂*8H₂O | Free acid | obtained | Yield* | Solubility | structure |
| Fumarate¹ | 2.044 g | 1.140 g | 0.999 g | 21% | Yes | No |
| Succinate | 2.098 g | 1.177 g | 0.958 g | 20% | Yes | Yes |
| L-ascorbate² | 2.094 g | 1.805 g | 2.005 g | 32% | Yes | No |
| L-glutamate | 2.017 g | 1.453 g | 0.175 g | 4% | Yes | Yes |
| Citrate | 2.057 g | 1.918 g | 1.123 g | 15% | Yes | Yes |
| L-Aspartate | 2.190 g | 1.316 g | 0.167 g | 3% | No | No |
| Tartrate | 2.070 g | 1.502 g | 2.005 g | 36% | Yes | Yes |

Notes
*Recovery calculated in % of the strontium content in Sr(OH)₂ *8H₂O and a stoichiometry that corresponds to the minimum content of the corresponding acid, e.g. a 1:1 ratio in the tartrate. The strontium salts of Table 7 (above) was characterised by powder x-ray crystallography and the corresponding diffractograms (not shown) showed that the products were relatively impure and of poor quality (i.e. heterogeneous crystal forms). Accordingly, the maximum yield of the room-temperature synthesis was evaluated to be 30%, which was calculated from the magnitude of characteristic peaks in the x-ray diffractograms. Weights were thus multiplied by a factor 0.3, as to obtain the estimated recovery and molecular weights of the strontium salts were used with the relevant amounts of bound crystal water. Although imprecise, the method reveals that the white powders of Table 7 did not contain high yields of the desired product. The remaining fraction of the product mainly consisted of unreacted reagents (i.e Strontium hydroxide) and strontium carbonate. If the strontium salts of Table 7 contained six water molecules in the crystal structure than the yield would be reduced even further by some 10-50%, as compared to the values presented. These estimates and difficulties in determination could result from formation substantial amounts of strontium carbonate when the salts were separated by re-crystallisation.
¹Fumaric acid is insoluble in water, and ethanol is added to the suspension until complete solubilization is achieved. The synthesis is continued with this material.
²In addition to the indicated amounts of strontium hydroxides and L-ascorbate an additional 4.087 g SrCl₂*6H₂O dissolved in water is added to the reaction mixture.

In conclusion, the methods known in the prior art literature, as exemplified in examples 8 & 9 for the preparation of strontium salts result in a relatively poor yield (at the most less than 40-50%). Furthermore, the data in this example demonstrates that strontium carbonate formation, heterogeneous crystal formation and presence of unreacted starting products in the reactant product is a general phenomenon when synthesizing strontium salts by methods disclosed in the prior art literature. In Examples 1-6 is given guidance for how to prepare strontium salt with a higher yield and high purity under mild conditions compatible with temperature and/or pH sensitive anions. The examples are intended for illustrative purposes and are not constructed to limit the invention in any way. Furthermore, a person skilled in the art can find guidance for preparation of other alkaline earth metal salts or metal-organic compounds of interest according to the present invention.

1-30. (canceled)

31. A strontium salt, which is strontium salicylate monohydrate with a unit cell crystal structure as depicted in FIGS. 1 and/or 2.

32. The strontium salt of claim 31, wherein the strontium salt is formed by a process comprising reacting strontium carbonate with salicylic acid in an aqueous medium to form a reaction mixture at a temperature of about 50° C. or less for a time period of at the most about 300 min.

33. The strontium salt of claim 32, wherein the process comprises adding the strontium carbonate in solid form with vigorous stirring and/or mixing to a solution of salicylic acid.

34. The strontium salt of claim 32, wherein the process further comprises monitoring the reaction mixture, and maintaining the pH of the reaction mixture below 9.5.

35. The strontium salt of claim 32, wherein the process comprises reacting the strontium carbonate with salicylic acid so that the ratio between the positive charges of strontium and the negative charges of the salicylate anion is 1:1.

36. The strontium salt of claim 32, wherein the process provides strontium salicylate mono-hydrate in a yield of 70% or more.

37. The strontium salt of claim 32, wherein the process provides strontium salicylate monohydrate without subsequent recrystallization in a purity of 80% or more.

38. The strontium salt of claim 32, wherein the process yields no more than about 1% of precipitated carbonate as compared with the amount of strontium salicylate monohydrate.

39. The strontium salt of claim 32, wherein the process comprises precipitating strontium salicylate monohydrate from the reaction mixture by adding about 5-60 vol/vol % alcohol to the reaction mixture.

40. The strontium salt of claim 39, wherein the alcohol is ethanol.

41. The strontium salt of claim 39, wherein the alcohol is methanol.

42. The strontium salt of claim 32, wherein the process comprises precipitating strontium salicylate monohydrate from the reaction mixture by adding about 5-60 vol/vol % acetone to the reaction mixture.

43. A method for the treatment and/or prophylaxis of a cartilage and/or bone disease and/or condition resulting in a dysregulation of cartilage and/or bone metabolism, comprising administering the strontium salt of claim 31 to a mammal in need thereof.

44. The method of claim 43, wherein the cartilage and/or bone disease and/or condition is osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, traumatic fracture, or atraumatic fracture.

45. A strontium salt, which is strontium malonate in crystalline form containing 1½ water molecule per crystal unit cell with a unit cell crystal structure as depicted in FIGS. 3 and/or 4.

46. The strontium salt of claim 45, wherein the strontium salt is formed by a process comprising reacting strontium carbonate with malonic acid in an aqueous medium to form a reaction mixture at a temperature of about 50° C. or less for a time period of at the most about 300 min.

47. The strontium salt of claim 46, wherein the process comprises adding the strontium carbonate in solid form with vigorous stirring and/or mixing to a solution of malonic acid.

48. The strontium salt of claim 46, wherein the process further comprises monitoring the reaction mixture, and maintaining the pH of the reaction mixture below 9.5.

49. The strontium salt of claim 46, wherein the process comprises reacting the strontium carbonate with malonic acid so that the ratio between the positive charges of strontium and the negative charges of the malonate anions is 1:1.

50. The strontium salt of claim 46, wherein the process provides strontium malonate sesquihydrate in a yield of 70% or more.

51. The strontium salt of claim 46, wherein the process provides strontium malonate sesquihydrate without subsequent recrystallization in a purity of 80% or more.

52. The strontium salt of claim 46, wherein the process yields no more than about 1% of precipitated carbonate as compared with the amount of strontium malonate sesquihydrate.

53. The strontium salt of claim 46, wherein the process comprises precipitating strontium malonate sesquihydrate from the reaction mixture by adding about 5-60 vol/vol % alcohol to the reaction mixture.

54. The strontium salt of claim 53, wherein the alcohol is ethanol.

55. The strontium salt of claim 53, wherein the alcohol is methanol.

56. The strontium salt of claim 46, wherein the process comprises precipitating strontium malonate sesquihydrate from the reaction mixture by adding about 5-60 vol/vol % acetone to the reaction mixture.

57. A method for the treatment and/or prophylaxis of a cartilage and/or bone disease and/or condition resulting in a dysregulation of cartilage and/or bone metabolism, comprising administering the strontium salt of claim 45 to a mammal in need thereof.

58. The method of claim 57, wherein the cartilage and/or bone disease and/or condition is osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, traumatic fracture, or atraumatic fracture.

59. A strontium salt, which is strontium di-ibuprofenate dihydrate with a unit cell crystal structure as depicted in FIGS. 7 and/or 8.

60. The strontium salt of claim 59, wherein the strontium salt is formed by a process comprising reacting strontium carbonate with ibuprofen in an aqueous medium to form a reaction mixture at a temperature of about 50° C. or less for a time period of at the most about 300 min.

61. The strontium salt of claim 60, wherein the process comprises adding the strontium carbonate in solid form with vigorous stirring and/or mixing to a solution of ibuprofen.

62. The strontium salt of claim 60, wherein the process further comprises monitoring the reaction mixture, and maintaining the pH of the reaction mixture below 9.5.

63. The strontium salt of claim 60, wherein the process comprises reacting the strontium carbonate with ibuprofen so that the ratio between the positive charges of strontium and the negative charges of the ibuprofenate anion is 1:1.

64. The strontium salt of claim 60, wherein the process provides strontium di-ibuprofenate dihydrate in a yield of 70% or more.

65. The strontium salt of claim 60, wherein the process provides strontium di-ibuprofenate dihydrate without subsequent recrystallization in a purity of 80% or more.

66. The strontium salt of claim 60, wherein the process yields no more than about 1% of precipitated carbonate as compared with the amount of strontium di-ibuprofenate dihydrate.

67. The strontium salt of claim 60, wherein the process comprises precipitating strontium di-ibuprofenate dihydrate from the reaction mixture by adding about 5-60 vol/vol % alcohol to the reaction mixture.

68. The strontium salt of claim 67, wherein the alcohol is ethanol.

69. The strontium salt of claim 67, wherein the alcohol is methanol.

70. The strontium salt of claim 60, wherein the process comprises precipitating strontium di-ibuprofenate dihydrate from the reaction mixture by adding about 5-60 vol/vol % acetone to the reaction mixture.

71. A method for the treatment and/or prophylaxis of a cartilage and/or bone disease and/or condition resulting in a dysregulation of cartilage and/or bone metabolism, comprising administering the strontium salt of claim 59 to a mammal in need thereof.

72. The method of claim 71, wherein the cartilage and/or bone disease and/or condition is osteoporosis, osteoarthritis, osteopetrosis, osteopenia and Paget's disease, hypercalcemia of malignancy, periodontal disease, hyperparathyroidism, periarticular erosions in rheumatoid arthritis, osteodystrophy, myositis ossificans, Bechterew's disease, malignant hypercalcemia, osteolytic lesions produced by bone metastasis, bone pain due to bone metastasis, bone loss due to sex steroid hormone deficiency, bone abnormalities due to steroid hormone treatment, bone abnormalities caused by cancer therapeutics, osteomalacia, Bechet's disease, hyperostosis, metastatic bone disease, immobilization-induced osteopenia or osteoporosis, or glucocorticoid-induced osteopenia or osteoporosis, osteoporosis pseudoglioma syndrome, idiopathic juvenile osteoporosis, traumatic fracture, or atraumatic fracture.
